SEQUENCE LISTING (4 sequences)

SEQ ID NO: 1 - protein, 17 aa, UNKNOWN
  nociceptin opioid-like peptide
FGGFTGARKSARKLANQ

SEQ ID NO: 2 - protein, 17 aa, UNKNOWN
  orphanin FQ2 opioid-like peptide
FSEFMRQYLVLSMQSSQ

SEQ ID NO: 3 - protein, 17 aa, UNKNOWN
  dynorphin A opioid-like peptide
YGGFLRRIRPKLKWDNQ

SEQ ID NO: 4 - protein, 13 aa, UNKNOWN
  residues 5-17 of noiceptin opioid-like peptide
TGARKSARKLANQ

(19) United States
(12) Patent Application Publication
(10) Pub. No.: US 2005/0266575 A1
(43) Pub. Date: Dec. 1, 2005

(54) Pharmacophores for nociceptin, methods of obtaining and using in screening for nociceptin mimics

(76) Inventors: Kevin H. Mayo, Minneapolis, MN (US); Mary Pat Beavers, New Hope, PA (US); Michael J. Orsini, Somerset, NJ (US); Steven A. Middleton, Flemington, NJ (US); Peter J. Connolly, New Providence, NJ (US)

Correspondence Address: MUETING, RAASCH & GEBHARDT, P.A., P.O. BOX 581415, MINNEAPOLIS, MN, 55458, US

(21) Appl. No.: 10/854,904

(22) Filed: May 27, 2004

(51) Int. Cl. G01N 33/00

(52) U.S. Cl. 436/86; 436/173

(57) ABSTRACT

Nociepeptin pharmacophores, methods of determining a nociceptin pharmocophore, nociceptin solution structures, and methods of identifying compounds as potential nociceptin mimics are provided.

### BACKGROUND

Nociceptin is a 17 amino acid (i.e., 17 mer) opioid-like peptide that was identified as a natural ligand of the orphan opioid receptor ORL1. It has also been called orphanin FQ to signify that it is a ligand for ORL1 with an amino-terminal phenylalanine (F) and a carboxy-terminal glutamine (Q). The sequence of nociceptin is H-Phe-Gly-Gly-Phe-Thr-Gly-Ala-Arg-Lys-Ser-Ala-Arg-Lys-Leu-Ala-Asn-Gln-OH (SEQ ID NO: 1).

Nociceptin signaling through ORL1 elicits many of the same responses induced by opioid signaling through the opioid receptors. Nociceptin causes inhibition of adenylyl cyclase, activation of potassium channels, inhibition of calcium channels, mobilization of intracellular calcium, and activation of mitogen-activated protein kinase.

These effects of this peptide indicate that, like opioids, nociceptin has an inhibitory effect on synaptic transmission in the nervous system, acting to reduce secretion of neurotransmitters. Consistent with its cellular effects, nociceptin inhibits the release of glutamate, GABA, acetylcholine, tachykinin, and noradrenaline neurotransmitters.

Based on their distribution in the brain and spinal cord, nociceptin and its receptor ORL1 may be involved in a wide range of functions, including learning, memory, attention, and emotion. Nociceptin is also implicated in various sensory processes, such as perception of pain, and visual, auditory and olfactory function.

Nociceptin is related to dynorphin A, a peptide 17 mer ligand of the K-opioid receptor. Dynorphin A also binds ORL1, but with 100-fold lower affinity than nociceptin. Orphanin FQ2 is another biologically active peptide 17 mer processed from the same nociceptin precursor, prenociceptin. FIG. 1 shows the amino acid sequences of orphanin FQ2 (F S E F M R Q Y L V L S M Q S S Q (SEQ ID NO:2)), nociceptin (F G G F T G A R K S A R K L A N Q (SEQ ID NO:1)), and dynorphin A (Y G G F L R R I R P K L K W D N Q (SEQ ID NO:3)).

Although nociceptin and dynorphin A are the most homologous of these three peptides (particularly at the N- and C-termini and being poly-cationic), there are clear sequential differences that should affect conformation and receptor binding as noted above. For example, dynorphin A contains a conformationally-constraining proline residue and has mid-segment cationic residues sequentially out-of-sink relative to nociceptin.

Knowledge of the structures of these peptides would be highly informative, and could aid in understanding their functions. However, both dynorphin A and nociceptin have been structurally elucive. In previous biophysical studies, Dynorphin A and nociceptin have shown little tendency to form well-defined structures in water and in other polar solvents, as well as in lower dielectric helix-inducing media.

### SUMMARY

The present invention relates to the NMR solution structure of nociceptin in membrane-like environments (e.g., trifluoroethanol (TFE) and sodium dodecyl sulfate (SDS) micelles), as well as methods of obtaining and using such structural information. This information is extremely valuable in understanding the design of small molecules that can mimic the structure, and preferably the function, of nociceptin.

For example, the solution structure has a relatively stable helix conformation in aqueous solution with SDS micelles. The conformation from residues (i.e., amino acids) 5 (G or Thr) to 17 (Q or Gln) with functionally important N-terminal residues are folded aperiodically on top of the helix and two pairs of alanine-arginine residues lie on top of one another. Structure-activity relationships allows identification of pharmacophore sites, which preferably include the side chain functionalities from the Phe-1 aromatic group (i.e., the phenyl ring of the phenylalanine at position 1 in the 17 mer), the Phe-4 aromatic group (i.e., the phenyl ring of the phenylalanine at position 4 in the 17 mer), and the Arg-8 terminal nitrogen atom of the guanidinium group (i.e., the terminal nitrogen atom of the guanidinium group of the arginine at position 8 in the 17 mer). These sites can be used in small molecule database searches, affording hits with nociceptin receptor binding affinities, and preferably with at least one nociceptin biological activity (referred to herein as nociceptin mimics).

Thus, as used herein, a nociceptin mimic is a compound that will bind to the receptor, and preferably function as a nociceptin receptor modulator. As such, the compound may function as an antagonist, agonist, or have other biological activity of that of nociceptin.

In one embodiment, the present invention provides a method of determining a nociceptin pharmocophore. The method includes: collecting nuclear magnetic resonance (NMR) data of nociceptin or a peptide analog thereof in an aqueous composition; determining distances and angles between atoms of nociceptin from the NMR data; computer modeling the three-dimensional structure of nociceptin in the aqueous composition based on the NMR data; and conducting a structure-activity analysis (e.g., an amino acid scan) to identify pharmacophore elements of the solution structure of nociceptin.

In another embodiment, the present invention provides a method of identifying a nociceptin mimic. The method includes: determining a nociceptin pharmocophore that includes: collecting nuclear magnetic resonance (NMR) data of nociceptin in an aqueous composition; determining distances and angles between atoms and features of nociceptin from the NMR data; computer modeling the three-dimensional structure of nociceptin in the aqueous composition based on the NMR data; and conducting a structure-activity analysis (e.g., an amino acid scan) to identify pharmacophore elements of the solution structure of nociceptin; supplying a three-dimensional structure of a test compound; comparing the structural features of a test compound to the pharmacophore to determine if it is a potential nociceptin mimic; and evaluating the binding capacity of the potential mimic to a nociceptin receptor, wherein a nociceptin mimic wherein a nociceptin mimic inhibits the binding of labeled ¹²⁵I-nociceptin to human nociceptin receptor (ORL-1) on HEK-293 cell membranes by 50% or more at a concentration of 10 μM.

The present invention also provides a method of identifying a compound that binds to a nociceptin receptor. The method includes: determining a nociceptin pharmocophore including: collecting nuclear magnetic resonance (NMR) data of nociceptin in an aqueous composition; determining distances and angles between atoms and features of nociceptin from the NMR data; computer modeling the three-dimensional structure of nociceptin in the aqueous composition based on the NMR data; and conducting a structure-activity analysis to identify pharmacophore elements of the solution structure of nociceptin; supplying a three-dimensional structure of a test compound; comparing the structural features of a test compound to the pharmacophore to determine if the test compound will potentially bind to a nociceptin receptor; and evaluating the binding capacity of the identified test compound to a nociceptin receptor.

Preferably, evaluating the binding capacity includes using computer modeling techniques to evaluate the potential ability of the potential mimic to bind to a nociceptin receptor. Evaluating the binding capacity can optionally further include subjecting the potential mimic to a binding assay.

In certain embodiments, collecting NMR data includes carrying out a multidimensional NMR experiment on the aqueous composition of nociceptin. Preferably, the multidimensional NMR experiment is a 2D homonuclear NMR experiment. Preferably, determining distances and angles from NMR data includes carrying out Nuclear Overhauser Effect (NOE) experiments.

In certain embodiments, the aqueous composition of nociceptin or a peptide analog thereof includes a membrane-like environment. Preferably, the membrane-like environment includes a hydrophobic compound. Typically, the hydrophobic compound is used in an amount sufficient to provide an aqueous composition having a dielectric constant of less than 40.

In certain embodiments, the nociceptin pharmacophore includes at least three elements having two hydrophobic features and one polar feature or a feature capable of electrostatic interaction. Preferably, the nociceptin pharmacophore includes at least three elements having two hydrophobic features and one positively charged feature or a feature capable of hydrogen bonding. Preferably, the hydrophobic features are aromatic rings. In one embodiment, the pharmacophore includes the side chain functionalities from the Phe-1 aromatic group, the Phe-4 aromatic group, and the Arg-8 terminal nitrogen atom of the guanidinium group.

Preferably, a three-dimensional structure of nociceptin is represented by the Cartesian coordinates listed in Table 4. In certain embodiments, a three-dimensional structure of a nociceptin pharmacophore is represented by the Cartesian coordinates listed in Table 5, and alternatively in Table 6.

The present invention also provides a solution conformation of nociceptin in a membrane-like environment, wherein nociceptin forms a helix, amino acids 5 through 17 of nociceptin are folded aperiodically on top of the helix such that two pairs of alanine-arginine residues lie on top of one another and two pairs of alanine-arginine residues lie on top of one another. Preferably, the phenyl ring of F1 is positioned on top of that for F4, which is positioned over A7 and R8. Preferably, the surface active domain includes amino acids F1, F4, R8, A7, R12, and A11 shown in FIG. 4.

The present invention also provides a solution conformation of nociceptin that includes a three-dimensional structure represented by the Cartesian coordinates listed in Table 4. A machine-readable data storage medium that includes a data storage material encoded with structure coordinates listed in Table 4 is also provided, as is a machine-readable data storage medium that includes a data storage material encoded with structure coordinates listed in Table 5, or alternatively, Table 6.

The terms “comprises” and variations thereof do not have a limiting meaning where these terms appear in the description and claims.

As used herein, “a,” “an,” “the,” “at least one,” and “one or more” are used interchangeably.

Also herein, the recitations of numerical ranges by endpoints include all numbers subsumed within that range (e.g., 1 to 5 includes 1, 1.5, 2, 2.75, 3, 3.80, 4, 5, etc.).

The term “ligand” as used herein means any chemical entity, compound, or portion thereof, that is capable of binding to a protein.

The term “binding” as used herein, refers to a condition of proximity between a chemical entity or compound, or portions thereof, and a target protein or portions thereof. The association may be non-covalent, wherein the juxtaposition is energetically favored by hydrogen bonding, van der Waals forces, or electrostatic interactions, or it may be covalent. The association may be a static interaction, or an equilibrium may be reached between associated and non-associated species.

“Amino acid” is used herein to refer to a chemical compound with the general formula: NH₂—CRH—COOH, where R, the side chain, is H or an organic group. Where R is an organic group, R can vary and is either polar or nonpolar (i.e., hydrophobic). The amino acids of this invention can be naturally occurring or synthetic (often referred to as nonproteinogenic). As used herein, an organic group is a hydrocarbon group that is classified as an aliphatic group, a cyclic group or combination of aliphatic and cyclic groups. The term “aliphatic group” means a saturated or unsaturated linear or branched hydrocarbon group. This term is used to encompass alkyl, alkenyl, and alkynyl groups, for example. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” refers to mono- or polycyclic aromatic hydrocarbon groups. As used herein, an organic group can be substituted or unsubstituted.

The terms “polypeptide” and “peptide” as used herein, are used interchangeably and refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or naturally occurring. This term also includes polypeptides that have been modified or derivatized, such as by glycosylation, acetylation, phosphorylation, and the like.

The following abbreviations are used throughout the application:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

The following abbreviations have been used herein: NMR, nuclear magnetic resonance spectroscopy; NOE, nuclear Overhauser effect; rf, radio frequency; FID, free induction decay; HPLC, high performance liquid chromatography.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1. Peptide sequences. Amino acid sequences are shown for nociceptin, orphanin FQ2 and dynorphin A.

FIG. 2. NOESY Spectra for Nociceptin in SDS. 600 MHz ¹H NMR spectra are shown for nociceptin in the absence (A) and presence (B) of SDS micelles. Peptide concentration was 6.3 mM in 10 mM potassium phosphate, 10 mM SDS, pH 5.5 and 25° C. Spectra were accumulated with 8 k data points over 6000 Hz sweep width and were processed with 1 Hz line broadening. 1H—NH and NH—NH spectral regions are shown and some key NOEs are identified.

FIG. 3. Summary of NOEs. Observed NOEs are summarized for nociceptin in SDS. The thickness of the bar is proportional to the magnitude of the NOE.

FIG. 4. NOE-Derived Structures of Nociceptin in SDS micelles. For nociceptin in SDS micelles, 14 final NOE-derived structures have been superimposed at the left of the figure and structural statistics are given in Table 2. One surface of the amphipathic helix is shown in the space-filling model and the average structure is also shown.

FIG. 5. Competitive Receptor Binding Assay. (A) Dose response curves for alanine substitutions at positions 1, 4 and 8 are compared to native nociceptin. (B) Dose response curves for targeted substitutions at positions 5, 7, 8, 11 and 12 are shown, with the specific change noted. See Table 3 for comparison of Ki values of other substitutions at these positions. Binding data was obtained and Kᵢvalues were calculated as described in Methods. (C) Correlation plot for receptor binding and calcium release. Nociceptin receptor binding and functional data (calcium release) are plotted versus one another. A standard linear fit gives a correlation coefficient of 0.84.

FIG. 6. Bar Graphs showing relative receptor binding activities and calcium release for nociceptin and its alanine-scanning variants. Data shown in panels A and B were calculated by taking the natural logarithm of the experimental value for an alanine-substituted peptide divided by that value for parent nociceptin. Data shown in panel C was calculated by taking the ratio in panel B divided by that in panel A. The amino acid sequence for nociceptin is given at the bottom of panel B.

FIG. 7. Chemical structures of compounds identified from 3D pharmacophore searches of chemical libraries demonstrating nociceptin receptor binding activity. Identification was based on inhibition of ¹²⁵I-nociceptin binding to human nociceptin receptor (ORL-1) on HEK-293 cell membranes by test compounds at a concentration of 10 μM (average of two determinations).

### DETAILED DESCRIPTION OF ILLUSTRATIVE EMBODIMENTS

The present invention relates to the NMR solution structure of nociceptin in membrane-like environments (e.g., TFE and SDS micelles), as well as methods of obtaining and using such structural information. Such structural information is used in determining a “pharmacophore,” which is an idealized, three-dimensional definition of the structural requirements for biological activity. That is, a pharmacophore defines the structural characteristics needed for a lead compound to specifically bind to a target of interest.

Such compounds that bind to a receptor at the same site as a ligand are often referred to as ligand “mimics.” This term can encompasses molecules having portions similar to corresponding portions of the ligand in terms of structure. Such compounds have the potential for having the same or similar biological function to that of the ligand. Preferably, such compounds also have similar biological function to that of the ligand. Such mimics can be peptides or small organic molecules. This finds significant utility in improving the lead compound determination in drug discovery processes because the compounds that share the common pharmacophore structure typically bind to a target molecule in a chemically and physically similar manner.

The most fundamental specification of a pharmacophore is in terms of both the chemical groups making up the pharmacophore and the geometric relationships of these groups. Several chemical arrangements may have similar electronic properties. For example, if a pharmacophore specification included an —OH group at a particular position, a substantially equivalent specification might include an —SH group at the same position. Equivalent chemical groups that may be substituted in a pharmacophore specification without substantially changing its nature are called homologous.

In particular embodiments, therefore, this invention provides a method for the determination of a pharmacophore, which includes the specification of the geometric relationships of chemical groups within a nociceptin molecule, for identifying nociceptin mimics that bind to a nociceptin receptor. The nociceptin pharmacophore according to the present invention is preferably determined by NMR techniques, including conformation analysis and derived NOEs, followed by structure-activity assays, particularly amino acid scans, of nociceptin and peptide analogs thereof.

Specifically, in the present invention, two-dimensional (2D) NMR conformation analysis was used to demonstrate that nociceptin has a propensity to fold as an alpha-helix. Derived NOEs were used to model the structure of the peptide in solution, and in particular the distances and angles between atoms.

A variety of solution NMR techniques can be used to determine structural relationships between atoms within a molecule. Thus, although 2D homonuclear NMR experiments were used for the experiments described herein, other multidimensional experiments can be used that are either homonuclear of heteronuclear. Furthermore, although NOE experiments were used to derive distances and angles between atoms in the experiments described herein, other NMR methods such as evaluation of coupling constants, H-D exchange experiments, and the like, can be used. Suitable techniques include the evaluation of residual coupling constants, the evaluation of the chemical shift index, etc.

In modern multidimensional NMR NOE experiments, excitation (i.e., perturbation) of a particular nucleus is performed at a range of frequencies simultaneously so that frequencies are read off axes in each dimension. The central frequency and frequency range is selected for a given nucleus type so that it will excite all of those nuclei in the molecule. As a result of perturbing the nucleus of a particular atom, the nuclei of other nearby atoms can be perturbed as well. The Nuclear Overhauser Effect (NOE) can cause detectible changes in the NMR signal of a nucleus that is proximal to the perturbed nucleus. The signal changes are the result of magnetization transfer to the proximal atoms. Since an NOE occurs by spatial proximity, not merely connection via chemical bonds, it is especially useful for determining distances between molecules or atoms within a molecule. The term “proximal” herein means within a defined distance of one or more atoms of interest, where the defined distance is a function of the method used to perturb. Functionally, “proximal” can be defined as being within a distance where perturbation can be detected. When NOE is used, the distance is usually 5 Angstroms. Such perturbation can be detected and identified by a variety of known methods. Detectable changes in NMR signals include changes in intensity (NOE), location (chemical shift) or width (linewidth). For example, nuclear Overhauser enhancement spectroscopy experiments (NOESY) can be used. In practice, the perturbed nuclei of atoms in large molecules can be identified using a multidimensional multinuclear NMR method to identify NMR cross-peaks corresponding to the perturbed atoms. Numerous other variant experiments and modifications are known in the art including heteronuclear NMR experiments, which are particularly useful with larger proteins. For example, two-dimensional NMR experiments can measure the chemical shifts of two types of nuclei and higher-dimensional NMR experiments can be used to measure the chemical shifts of additional types of nuclei and to eliminate problems with cross peak overlap if spectra are too crowded.

Structures can be derived from the NMR derived information (e.g., interatom distance contraints, hydrogen bond constraints, bonding and nonbonding angles) using various computer programs (e.g., X-PLOR). Typically such computer programs are based on molecular dynamics and energy minimizations including evaluations of non-covalent interactions between atoms such as hydrogen bonding, van der Waals forces, or electrostatic interactions.

Structure-activity relationships (SAR) can be evaluated using a number of assays, in addition to the amino acid scans used herein. For example, site directed mutagenesis can be used as well as combinatorial methods that provide systematic changes in structure. The activity of these analogs (e.g., systematically structurally modified molecules) are then evaluated and compared. Such activities typically include binding to the receptor, but can also include a wide variety of other biological activies depending on the biological function of the model compound (e.g., nociceptin in the present application). In the present invention, an alanine scan (i.e., alanine walk through) was performed on the nociceptin sequence in which each amino acid was substituted sequentially by alanine (or glycine if the amino acid being replaced was alanine). Various other substitutions were made at various residues using various amino acids to determine changes in charge, polarity, and side-chain composition. This provided information about the portions of the molecule that are involved in binding and biological function (e.g., calcium release). Similar information can be obtained using a variety of SAR techniques.

In general, a pharmacophore is specified by the precise electronic properties on the surface of the binder (i.e., ligand) that causes binding to the surface of the target molecule (i.e., receptor). Typically, these properties are specified by the underlying, causative, chemical structures (e.g., aromatic groups, functional groups such as —COOH, etc.). The preferred pharmacophore representation consists of a specification of the underlying chemical groups and their geometric relations. The more precisely the geometric relations are specified, the more preferred. In preferred but not limiting aspects, the geometric relations are precise to at least 2 Angstroms, and most preferably, at least 1 Angstrom. A pharmacophore will usually include the identification of 2 to 4 of such groups (i.e., pharmacophoric elements or features), with 3 being typical. However, for complex protein recognition targets, a pharmacophore may include a greater number of groups.

In the present invention, the solution structure of native nociceptin was determined by NMR techniques, including 2D-homonuclear magnetization transfer (HOHAHA) and NOESY experiments. The solution structure has a relatively stable helix conformation in aqueous solution. This structure can be obtained using a relatively low-dielectric aqueous composition, which provides a membrane-like environment. Typically, such an environment can be provided by adding hydrophobic compounds, such as trifluoroethanol, sodium dodecyl sulfate, dodceyl phosphatidyl choline. Such compounds are preferably used in amounts sufficient to provide an aqueous composition having a dielectric constant of less than 40.

Thus, the present invention provides nociceptin in a membrane-like environment defined by NMR data.

The conformation from residues 5 to 17 (i.e., T G A R K S A R K L A N Q (SEQ ID NO:4)) of nociceptin, including functionally important N-terminal residues, are folded aperiodically on top of the helix. Furthermore, two pairs of alanine-arginine residues lie on top of one another. The structure further includes the phenyl ring of F1 positioned on top of that for F4, which is positioned over A7 and R8.

In functional assays for receptor binding and calcium flux, amino acid scanning variants nociceptin indicated that functionally-key residues generally followed helix periodicity. That is, alanine-containing and other amino acid analogs of the nociceptin 17 mer were probed in order to identify residues that were important to bioactivity. See Table 3. These functional assays indicated the structure to be in a helix conformation, which is consistent with the NMR structural model.

The pharmacophore sites of nociceptin according to the present invention include some combination of the following elements: two hydrophobic features (which can be either straight chain or branched alkyl groups, either an aryl or heteroaryl system or a fused aromatic or heteroaromatic system, for example); and one polar feature or a feature capable of electrostatic interaction (preferably, one positively charged feature or a feature that is capable of hydrogen bonding (i.e., containing either an H-bond donor, an H-bond acceptor, or both)). The distances in Angstroms between pharmacophoric elements correspond to the distances between pharmacophoric elements (amino acid side chain functionalities) in the nociceptin peptide NMR structure. Specifically, the elements of the most preferred pharmacophore include features represented by the side chain functionalities from the Phe-1 aromatic group (i.e., the phenyl ring of the phenylalanine at position 1 in the 17 mer), the Phe-4 aromatic group (i.e., the phenyl ring of the phenylalanine at position 4 in the 17 mer), and the Arg-8 terminal nitrogen atom of the guanidinium group (i.e., the terminal nitrogen atom of the guanidinium group of the arginine at position 8 in the l7 mer). These sites can be used in small molecule database searches affording hits with demonstrated nociceptin receptor binding affinities.

Thus, the present invention provides a pharmacophore for nociceptin generated in accordance with the methods of the present invention. The pharmacophore of the invention can be defined by Cartesian coordinates, x, y and z, which represent distances between the centroid of the aromatic ring features, and selected atoms of the SAR-important side chain functionalities.

The 3D queries can include features such as atoms, lines, planes, centroids, extension points, hydrogen bond sites, and hydrophobic sites. The geometric relationships between pharmacophore features can be defined using distance, angle, and volume constraints. The Cartesian coordinates of the 3D structures of two representative pharmacophores are listed in Tables 5 and 6.

The coordinates of the pharmacophore of the invention define the relative relationship between the elements (i.e., features), and therefore, those of skill in the art will recognize that the specific coordinates are dependent upon the specific coordinate system used, and thus, although rotation or translation of these coordinates may change the specific values of the x, y and z coordinates, the coordinates will define the claimed model. Those skilled in the art will also recognize that the pharmacophore of the invention may encompass any model, after optimal superposition of the models, comprising the identified features and having a root mean square deviation of equivalent features of less than 3.0 Angstroms. More preferably, the pharmacophore of the invention encompasses any model comprising the identified features and having a root mean square deviation of equivalent features of less than 2.0 Angstroms, even more preferably less than 1.5 Angstroms, and most preferably, less than 1.0 Angstrom. The term “root mean square deviation” means the square root of the arithmetic mean of the squares of the deviations.

Thus, the present invention provides molecules including the pharmacophore elements, as defined by the 3D structural coordinates listed in Tables 5 and 6. Additional pharmacophores may be derived from the 3D structure of the nociceptin peptide (represented by the 3D structural coordinates listed in Table 3), using the structure activity information for the corresponding nociceptin peptide.

Those of skill in the art understand that a set of structure coordinates define a relative set of points that, in turn, define a configuration in three dimensions. A similar or identical configuration can be defined by an entirely different set of coordinates, provided the distances and angles between coordinates remain essentially the same. In addition, a scalable configuration of points can be defined by increasing or decreasing the distances between coordinates by a scalar factor while keeping the angles essentially the same. The configurations of points in space derived from structure coordinates according to the invention can be visualized as, for example, a holographic image, a stereodiagram, a model, or a computer-displayed image, and the invention thus includes such images, diagrams or models.

The present invention also provides molecules that contain one or more structural features that are similar to the structural features of nociceptin. These molecules are referred to herein as “structurally homologous” to nociceptin. Similar structural features can include, for example, regions of amino acid identity, conserved binding site motifs, and similarly arranged secondary structural elements (e.g., α helices, β sheets, turns). Examples of such molecules include the peptide analogs disclosed in Table 3. Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Preferably, two amino acid sequences are compared using the Blastp program, version 2.0.9, of the BLAST 2 search algorithm, as described by Tatusova et al., FEMS Microbiol Lett., 174:247-50 (1999), and available on the world wide web at ncbi.nlm.nih.gov/gorf/bl2.html. Preferably, the default values for all BLAST 2 search parameters are used, including matrix =BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.” Preferably, a structurally homologous molecule is a peptide that has an amino acid sequence sharing at least 65% identity with the amino acid sequence of nociceptin (SEQ ID NO: 1).

The invention further provides a machine-readable storage medium including a data storage material encoded with structure coordinates which, when using a machine programmed with instructions for using said data, displays a graphical three-dimensional representation of any of the molecules or pharmacophores of this invention. These structure coordinates are presented in Table 4 for a nociceptin structure, and in Tables 5 and 6 for two pharmacophore structures. This information can be used to screen compound libraries to determine if a compound is a potential nociceptin mimic (i.e., a potential binder to a nociceptin receptor) by determining if the features of the test compounds (i.e., compounds in the library) can adopt the necessary three-dimensional arrangement to fit the nociceptin pharmacophore model.

A system for reading a data storage medium may include, for example a computer including a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid cyrstal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers). The system may also include additional computer controlled devices such as consumer electronics and appliances. Input hardware may be coupled to the computer by input lines and may be implemented in a variety of ways (e.g., via the use of a modem or modems connected by a telephone line or dedicated data line, CD-ROM drives or disk drives). Output hardware may be coupled to the computer by output lines and may similarly be implemented by conventional devices (e.g., the output hardware may include a display device for displaying a graphical representation, a printer, or a disk drive to store system output for later use). In operation, a CPU coordinates the use of the various input and output devices, coordinates data accesses from mass storage devices, accesses to and from working memory, and determines the sequence of data processing steps.

A number of programs may be used to process the machine-readable data of this invention. Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

The three-dimensional (3D) pharmacophore derived from the structural information for nociceptin and its peptide analogs provides a starting point for electronic database searching. Both corporate and commercial libraries can be searched against the 3D pharmacophore to identify lead chemical structures in drug design work. That is, compounds are selected from such libraries that structurally resemble the 3D-pharmacophore. In the present invention, identifying compounds that have the desired structural features of the nociceptin 3D-pharmacophore include those with functionalities analogous to the Phe-1 aromatic group, the Phe-4 aromatic group, and the Arg-8 terminal nitrogen atom of the guanidinium groups.

The databases of libraries can first be filtered to remove any molecules whose molecular weights are too high, since high molecular weights can render the compounds unsuitable for further development as drugs, particularly oral drugs. Thus, the initial filtration to remove molecules whose molecular weights are too high preferably involves evaluating compounds having a molecular weight of no more than 1000 Daltons and more preferably no more than 500 Daltons.

This subset of lower molecular weight compounds (i.e., test compounds) can be evaluated for the desired structural features analogous to the nociceptin 3D-pharmacophore. Computational techniques can be used to screen, identify, select, and design structurally analogous chemical entities. The degree of fit of a particular compound structure to the pharmacophore is calculated by determining, using computer methods, if the compound possesses the chemical features of the model and if the features can adopt the necessary three-dimensional arrangement to fit the model. Typically, this fitting process involves maximizing the interfacial or intersurface contact between the ligand and receptor.

The software employed in the 3D search will allow for a conformational search of each molecule in the electronic database, and those conformers that contain the necessary chemical features with the proper distances between pharmacophore elements will be defined as “fitting” the pharmacophore.

The feature search and the geometric search will both be done during the flexible search routine.

Alternatively, many conformations (as many as 500 per molecule) can be stored as discreet molecules and the resulting multi-conformer database can then be searched to find molecules that fit the pharmacophore.

The Unity software provides a fitness score that can be used to rank the hits. This score is based on strain energy, RMSD of the features in hits to those in the pharmacophore hypothesis, and the number of rotatable bonds.

This can involve a comparison of three-dimensional structure, hydrophobicity, steric bulk, electrostatic properties, bond angles, size or molecular composition, etc. For example, Quanta's Molecular Similarity package (Molecular Simulations Inc., Waltham, Mass.) permits comparison between different structures, different conformations of the same structure, and different parts of the same structure. Typically, the structure of the compound being analyzed is subjected top a comformational search and a feature search during the 3D flex search routine. Alternatively, a multi-conformational electronic database can be prepared and in this way, the conformers of each molecule are stored prior to the feature search. The modeling program will select those compounds having the proper conformational arrangement of pharmacophoric elements. Examples of other suitable computer programs include UNITY (Tripos, Inc.), and Catalyst (Accelrys, Inc.).

In particular, computational techniques can be used to identify or design chemical entities that associate with a nociceptin binding pocket, and are potential inhibitors, agonists (e.g., which demonstrate calcium release), or antagonists (e.g., which do not demonstrate calcium release). Potential modifiers may bind to or interfere with all or a portion of the active site of a nociceptin receptor, and can be competitive, non-competitive, or uncompetitive inhibitors; or interfere with dimerization by binding at the interface between the two monomers. Once identified and screened for biological activity, these inhibitors/agonists/antagonists may be used therapeutically or prophylactically to treat pain, asthma, and/or anxiety, for example. Thus, such chemical entities are potential drug candidates.

To be a viable drug candidate, the chemical entity (herein, the nociceptin mimic) identified or designed according to the method must be capable of structurally associating with at least part of a nociceptin binding pocket in a receptor, and must be able, sterically and energetically, to assume a conformation that allows it to associate with a nociceptin binding pocket.

Preferred candidate structures are those having a set of structure coordinates for the identified features with a root mean square deviation (i.e., the square root of the arithmetic mean of the squares of the deviations of the mean) of conserved residue atoms of less than 3.0 Angstroms when superimposed on the relevant structure coordinates. More preferably, the root mean square deviation is less than 2.0 Angstroms, even more preferably less than 1.5 Angstroms, and most preferably, less than 1.0 Angstrom.

Optionally, the potential binding of a chemical entity to nociceptin binding pocket (i.e., a binding pocket of a nociceptin receptor) can be analyzed using computer modeling techniques prior to the actual synthesis and/or testing of the chemical entity. If these computational experiments suggest insufficient interaction and association between it and the nociceptin binding pocket, testing of the entity is obviated. However, if computer modeling indicates a strong interaction, the molecule may then be synthesized and tested for its ability to bind to or interfere with a nociceptin binding pocket. Binding assays are well known in the art and may employ kinetic or thermodynamic methodology using a wide variety of techniques including, but not limited to, microcalorimetry, isothermal denaturation, circular dichroism, capillary zone electrophoresis, nuclear magnetic resonance spectroscopy, fluorescence spectroscopy, and combinations thereof.

Typically, a nociceptin mimic inhibits the binding of labeled ¹²⁵I-nociceptin to its receptor by 50% or more at a concentration of 10 μM. A preferred nociceptin mimic has a binding capacity represented by a Kᵢ value of within 10% of that of nociceptin under the same conditions. A more preferred nociceptin mimic has a binding capacity represented by a Kᵢ value of within 5% of that of nociceptin under the same conditions.

In a preferred embodiment, a method of identifying a nociceptin mimic includes: determining a nociceptin pharmocophore that includes: collecting nuclear magnetic resonance (NMR) data of nociceptin in an aqueous composition; determining distances and angles between atoms and features (such as centroids of aromatic rings) of nociceptin from the NMR data; computer modeling the three-dimensional structure of nociceptin in the aqueous composition based on the NMR data; and conducting a structure-activity analysis to identify pharmacophore elements of the solution structure of nociceptin. This is followed by supplying a three-dimensional structure of a test compound; comparing the structural features of a test compound to the pharmacophore to determine if the features of the test compound can adopt the necessary three-dimensional arrangement to fit the model; and evaluating the binding capacity of the potential mimic to a nociceptin receptor, wherein a nociceptin mimic inhibits the binding of labeled ¹²⁵I-nociceptin to human nociceptin receptor (ORL-1) on HEK-293 cell membranes by 50% or more at a concentration of 10 μM.

As used herein, if the distance between a set of pharmacophore elements in the electronic hit is within 3.0 Angstroms of that corresponding distance in the pharmacophore hypothesis, this means that the test compound “fits” the model and is a potential mimic. Typically, there are at least three sets of distances for each pharmacophore hypothesis, and the tolerance in Angstroms may vary between one half and two Angstroms, with 1.0 Angstrom being a most preferred distance constraint. Each new pharmacophore query can specify a different distance constraint for each pair of features.

Once a candidate compound is identified using molecular modeling or database comparison techniques, it can be synthesized by a variety of techniques known to those of skill in the art.

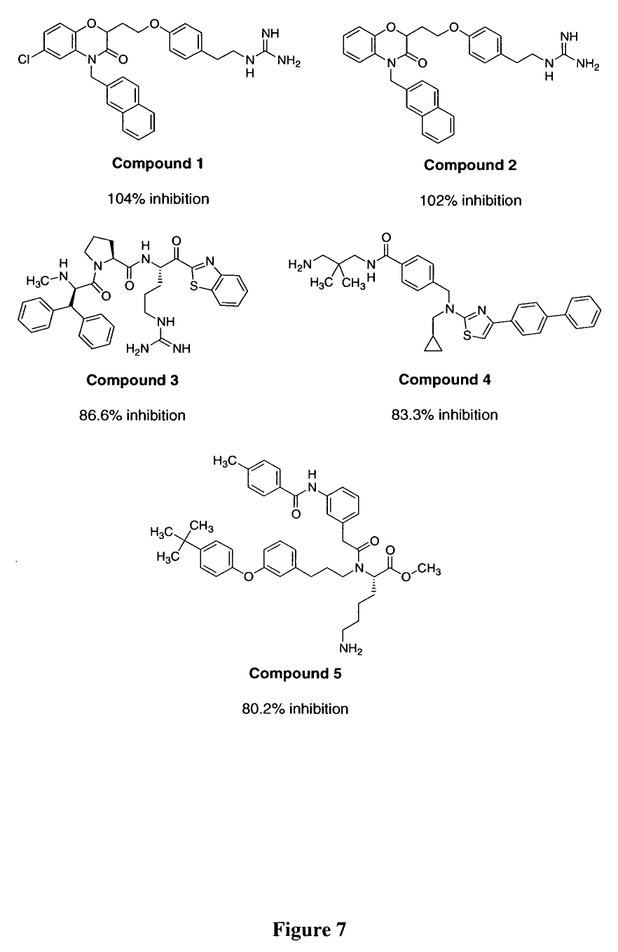

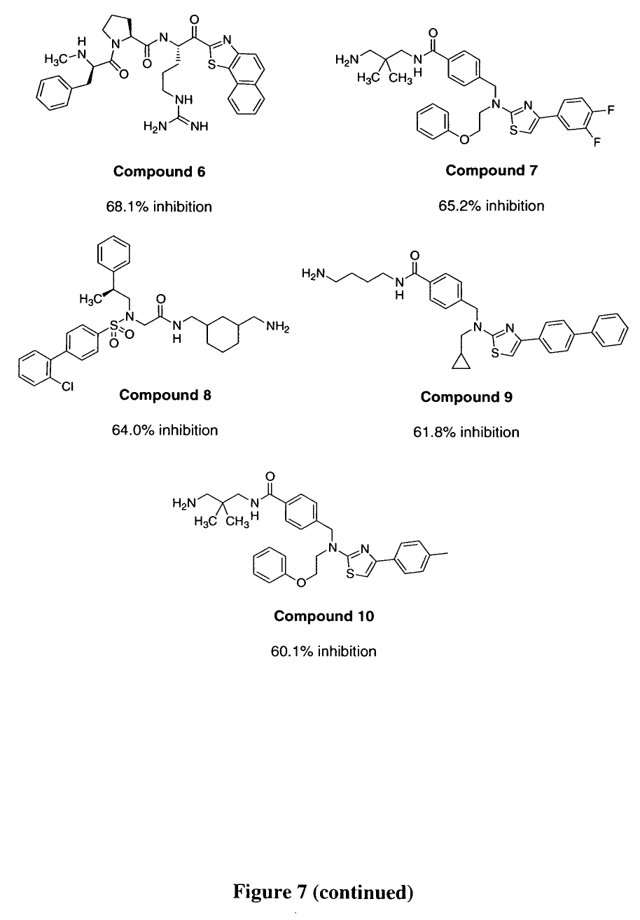

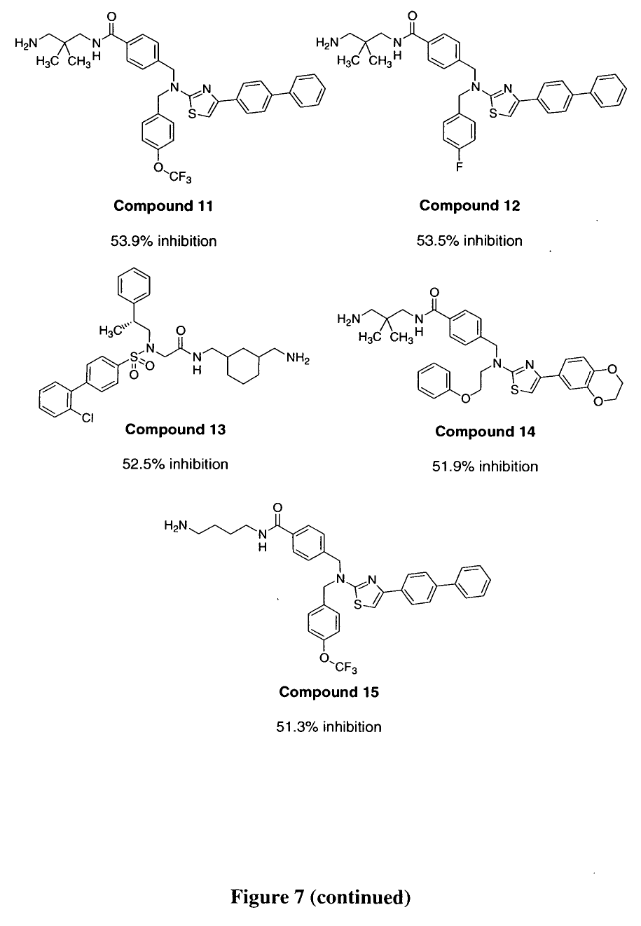

Examples of identified compounds that have the desired structural features of the nociceptin 3D-pharmacophore include those shown in FIG. 7. These compounds have the three basic pharmacophoric elements present in the native nociceptin 17 mer. These are the two phenyl rings of amino acids Phe-1 and Phe-4 of the positively charged guanidinium group of Arg-8.

These compounds, however, are greatly reduced in size compared to nociceptin. Nociceptin and its peptide analogs listed in Table 3 have molecular weights above 3000 grams/mole; the compounds identified in FIG. 7 having structural similarity to the nociceptin 3D-pharmacophore (and preferably having nociceptin receptor binding affinities) and relatively low molecular weights.

Once compounds are identified having analogous structural features to that of the nociceptin pharmacophore, and hence, to nociceptin, they are typically further screened to determine their function. In particular, a binding assay can be used. Compounds shown in FIG. 7 were tested in a nociceptin receptor binding assay, which indicated that they possess a function similar to that of nociceptin, at least with respect to binding. This demonstrates that a binding site on nociceptin can be reproduced in a small molecule, and that the entire length of the peptide is not required for binding, as long as the structure of the small molecule can adopt a conformation that mimics the conformation found in the biologically relevant section of the peptide.

### EXAMPLES

Objects and advantages of this invention are further illustrated by the following examples, but the particular materials and amounts thereof recited in these examples, as well as other conditions and details, should not be construed to unduly limit this invention.

Methods & Materials

Peptide preparation. Peptides were synthesized using a Milligen/Biosearch 9600 peptide solid-phase synthesizer using fluorenylmethoxycarbonyl chemistry. Lyophilized crude peptides were purified by preparative reversed-phase HPLC on a C18 column with an elution gradient of 0-60% acetonitrile with 0.1 % trifluoroacetic acid in water. Purity and composition of the peptides were verified by HPLC (Beckman Model 6300), amino acid analysis and mass spectrometry.

NMR Measurements. For NMR measurements, freeze-dried peptide was dissolved in H₂O. Peptide concentration was usually about 5 mM. pH was adjusted to pH 5.5 by adding microliter (μL) quantities of NaOD or DCl to the peptide sample. NMR spectra were acquired on a Varian UNITY Plus-600 NMR spectrometer.

2D-homonuclear magnetization transfer (HOHAHA) spectra, obtained by spin- locking with a MLEV-17 sequence with a mixing time of 60 ms, were used to identify spin systems (Bax et al., J. Magn. Reson., 65, 355-360 (1985)). NOESY experiments were performed for conformational analysis (Wider et al., J. Magn. Reson., 56, 207-234 (1984)). All 2D-NMR spectra were acquired in the States-TPPI phase sensitive mode (States et al., J. Magn. Res., 48, 286-292 (1982); Bodenhausen et al., J. Magn. Res., 37, 93-99 (1980)). The water resonance was suppressed by direct irradiation (0.8 second (s)) at the water frequency during the relaxation delay between scans as well as during the mixing time in NOESY experiments. 2D-NMR spectra were collected as 256 to 512 t1 experiments, each with 2k complex data points over a spectral width of 6 kHz in both dimensions with the carrier placed on the water resonance. For HOHAHA and NOESY spectra, 16 scans were time averaged per t1 experiment. Data were processed directly on the spectrometer or offline using VNMR (Varian, Inc., Palo Alto) or NMRPipe on an SGI workstation (Delaglio et al., J. Biomol. NMR, 6, 277-293 (1995)). Data sets were multiplied in both dimensions by a 30 to 60 degree shifted sine-bell function and zero-filled to 1k in the t1 dimension prior to Fourier transformation.

Pulsed field gradient (PFG) NMR self-diffusion measurements were performed as a check for peptide aggregation. PFG-NMR experiments were done as described by Mayo et al., Protein Sci., 5, 1301-1315 (1996), using a Varian Unity-Plus 500 NMR spectrometer. The maximum magnitude of the gradient was 60 G/cm, and the PFG longitudinal eddy-current delay pulse-sequence was used for all self-diffusion measurements which were performed in D20 at temperatures of 5° C. and 40° C. Peptide concentrations ranged from 0.1 mM to 15 mM. PFG NMR data were analyzed also as described by Mayo et al., Protein Sci., 5, 1301-1315 (1996).

Structural Modeling. Analysis of NOE growth curves indicated that backbone to backbone inter-proton NOEs were normally maximum at 300 milliseconds (ms) to 400 ms. Interproton distance constraints were derived from NOEs assigned in ¹H NOESY spectra acquired with mixing times of 200 ms and 400 ms. NOEs were classified as strong, medium, weak or very weak corresponding to upper bound distance constraints of 2.8, 3.3, 4.0, and 4.5 A, respectively. The lower bound restraint between non-bonded protons was set to 1.8 Å. Pseudo-atom corrections were added to the upper bound distance constraints where appropriate, and a 0.5 Å correction was added to the upper bound for NOEs involving methyl protons. Hydrogen bond constraints were identified from the pattern of sequential and interstrand NOEs involving NH and CαH protons, together with evidence of slow amide proton-solvent exchange. Each hydrogen bond identified was defined using two distance constraints; r_{NH-O}=1.8 to 2.5 Å, and r_{N-O}=1.8 to 2.5 Å.

Derived internuclear distance constraints were used in calculating structures for nociceptin by using X-PLOR (Brunger, X-plor Manual, Yale University Press, New Haven (1992)). The nociceptin structures were created using parallhdg.pro force fields. A template coordinate set was generated by using the Template routine. The ab initio simulated annealing (SA) protocol was then used. The SA procedure ran high temperature dynamics (3000 K for 120 picoseconds (ps)) and then cooled down to 100 K in 50 K steps with 1.5 ps molecular dynamics at each step. Powell minimization was performed at 100 K for 1000 steps. Structure refinement was done based on simulated annealing starting at 1000 K and ending at 100 K. Final structures were subjected to the X-PLOR Accept routine with the violation threshold for NOEs of 0.5 Å and dihedral angles of 5°. Angles, bond lengths or impropers were not allowed to deviate from ideal geometry more than 5°, 0.05 Å and 5°, respectively. Structures were superimposed using the BIOSYM INSIGHT viewer (Molecular Simulations, Inc.) and were analyzed using X-PLOR analysis routines.

Binding of peptides to the ORL-1 Receptor. The nociceptin receptor binding assay measures the binding of ¹²⁵I-Tyr¹⁴-nociceptin (2200 Ci/mmol, New England Nuclear) to human nociceptin receptor (ORL-1) on HEK-293 cell membranes. HEK-293 cell membranes from cells stably expressing the nociceptin receptor were prepared as described (Pulito et al., J. Pharmacol. Exp. Ther., 294, 224-229 (2000)), with the exception that the binding buffer used was 50 millimolar (mM) Tris-Cl pH 7.8, 5 mM MgCl₂ and 1 mM EGTA. ORL-1 membranes were diluted in binding buffer such that 25 μL containing 1 microgram (μg) of membrane was added to each well of a standard 96-well microtiter in binding buffer. Peptides were diluted at 50×the indicated concentration, and 1 μL was added to each well. ¹²⁵I-Tyr¹⁴-nociceptin was added at a final concentration of 0.5 nM so that the total reaction volume was 50 μL. Plates were incubated for two hours at room temperature and the reactions were filtered over GF/C Filterplates (Perkin Elmer-NEN), prewetted in 0.03% polyethleneimine, in a Filtermate 196 apparatus (Packard). Plates were washed six times with binding buffer in the filtration apparatus, then dried under vacuum for 1-2 hours. Twenty five microliters (25 μL) Microscint 20 (Packard) per well was added to solubilize bound radioactivity. The plates were then sealed and counted on a Packard Top Count to determine radioactivity bound to the membranes. Curves were fitted and IC₅₀ values determined using Graphpad Prizm software (v3.0).

For each peptide, the total binding was measured at several concentrations and the IC₅₀ (the concentration at which 50% of the binding is inhibited) was determined from the graphical display of X=logarithm of concentration vs. Y=response, using the following calculation:

$$Y = ( Minimum ) + \frac{( Maximum - Minimum )}{( 1 + {10}^{log ( {EC}_{50} - X )} )}$$

Calcium Flux. Although the ORL-1 receptor is coupled to Gᵢ, and thus does not nomally elicit a calcium response, cells were able to be pretreated with the muscarinic receptor agonist carbachol, upon which cells were “sensitized” and able to elicit a subsequent calcium response upon nociceptin administration. This technique has previously been described for nociceptin specifically (Connor et al., Br. J. Pharmacol., 119, 1614-1618 (1996) or Connor et al., Br. J. Pharmacol., 118, 205-207 (1996)) and for Gᵢ-coupled receptors in general (Chan et al., Mol. Pharmacol., 57, 700-708 (2000)).

HEK-293 cells expressing ORL-1 receptor were plated at a density of 50,000 cells/well in a total volume of 50 ↑L onto 96-well plates. Two days later cells were prepared for assay using the FLIPR Calcium Assay Kit (Molecular Devices) according to manufacturer's directions, with the exception that the volume of dye mix added to each well was 50 μL instead of 100 μL. Cells were then treated with 100 μM carbachol in 100 μL. After baseline was reestablished, cells were challenged with varying concentrations of nociceptin or a nociceptin variant. Data points were collected at one per second for 120 seconds, then one every three seconds for 30 seconds for a total collection time of 150 seconds after both the first and second additions.

Calcium flux as a result of nociceptin administration was calculated and divided by the response resulting from carbachol administration to normalize within individual wells. This produced a “percent of carbachol response” for each dose. The experiment was repeated 6 times and the high and low values for each concentration were dropped. The data were used to generate EC₅₀ curves using GraphPad Prizm v.3.0 software.

3D Database Searching. 3D Flex searches were performed with Unity 4.2 (Tripos, Inc., St. Louis, Mo.) using the Directed Tweak algorithm. Three point queries were defined for explicit atom types within the peptide pharmacophore using distance constraints with tolerances of ± one to two angstroms. Standard settings were used for the flexible searching. The Unity database was created from the command line starting from a Concord-generated 3D sd file.

Results & Discussion

NMR Conformational Analysis. In water, nociceptin exhibits a conformational distribution that is composed mostly of random coil with some highly transient helix structure (Salvadori et al., Biochem. Biophys. Res. Commun., 233, 640-643 (1997)). This is consistent with what was found for nociceptin dissolved in water alone (data not shown). However, nociceptin functions by interacting with a membrane receptor, and may infact do so by first interacting with membrane lipids (Schwyzer, Biochemistry, 25, 4281-4286 (1986)). Therefore, nociceptin in a relatively low dielectric environment, i.e., 30% (v/v) trifluoroethane (TFE) and water, and in the presence of SDS micelles was investigated. Both systems are often employed in NMR investigations to mimic membrane-like environments. In aqueous TFE, a series of NH—NH NOEs running from T5 to Q17 was observed that suggests the presence of nascent helix formation (Dyson et al., J. Mol. Biol., 201, 201-217 (1988)). Moreover, several αH—NH i,i+2 and i,i+3 NOEs were also identified, along with three NH—NH i,i+2 and two NH—NH i,i+3 NOEs. These NOEs were not observed in water alone and support the presence of multiple turn or helix conformation (Wüthrich et al., NMR of Proteins and Nucleic Acids, Wiley-Interscience, NY (1986); Wishart et al., Biochemistry, 31, 1647-1651 (1992)) that appears to be stabilized in a lower dielectric medium.

In the presence of SDS micelles, however, the peptide exhibits considerably more stable helical structure. This is first evidenced by differences in chemical shifts of resonances from the peptide, compared to those observed in aqueous TFE (Table 1).

TABLE 1
Secondary chemical shifts for nociceptin in SDS micelles. (Listed here
is the difference TFE minus SDS; a minus sign indicates a downfield
chemical shift when the peptide in the SDS)

| | NH | αH | βH | other |
|---|---|---|---|---|
| F1 | — | +0.55 | — | 2,6 +0.04 |
| | | | | 3,5 +0.06 |
| G2 | +0.28 | +0.1 | — | — |
| G3 | +1.0 | +0.2, +0.05 | — | — |
| F4 | +0.26 | +0.1 | +0.05, −0.2 | 2,6 −0.03 |
| | | | | 3,5 −0.03 |
| T5 | −0.02 | +0.1 | 0 | γH₃ 0 |
| G6 | −0.58 | −0.4 | — | — |
| A7 | +0.12 | +0.2 | (+0.05) | — |
| R8 | +0.1 | +0.25 | (0) | all (0) |
| K9 | +0.39 | +0.3 | (0) | all (0) |
| S10 | +0.17 | +0.2 | +0.05, 0 | — |
| A11 | +0.36 | +0.15 | (+0.05) | — |
| R12 | +0.34 | +0.2 | (0) | all (0) |
| K13 | +0.31 | +0.25 | (0) | all (0) |
| L14 | +0.34 | +0.2 | (−0.05) | all about 0 |
| A15 | +0.33 | +0.1 | (−0.05) | — |
| N16 | +0.25 | +0.1 | +0.15, 0 | — |
| Q17 | +0.01 | +0.2 | +0.1, +0.05 | γH₂ +0.05 |

Most NH and αCH resonances shift upfield and G3 αCH₂ and some αCH₂ resonances become either non-degenerate or better separated. The G3 αCH resonances, nearly degenerate in TFE, become separated by 0.25 parts per million (ppm) and upfield shifted by 0.68 ppm in SDS micelles. Relatedly, the G6 αCH₂ resonances are 0.87 ppm downfield shifted. These observations are probably due to ring current shifting from proximity to F1 and/or F4 and indicate structural stabilization and positioning of the phenylalanine ring either above the G3 αCH₂ group or on edge with the G6 αCH₂ group. Moreover, numerous structurally informative NOEs are observed.

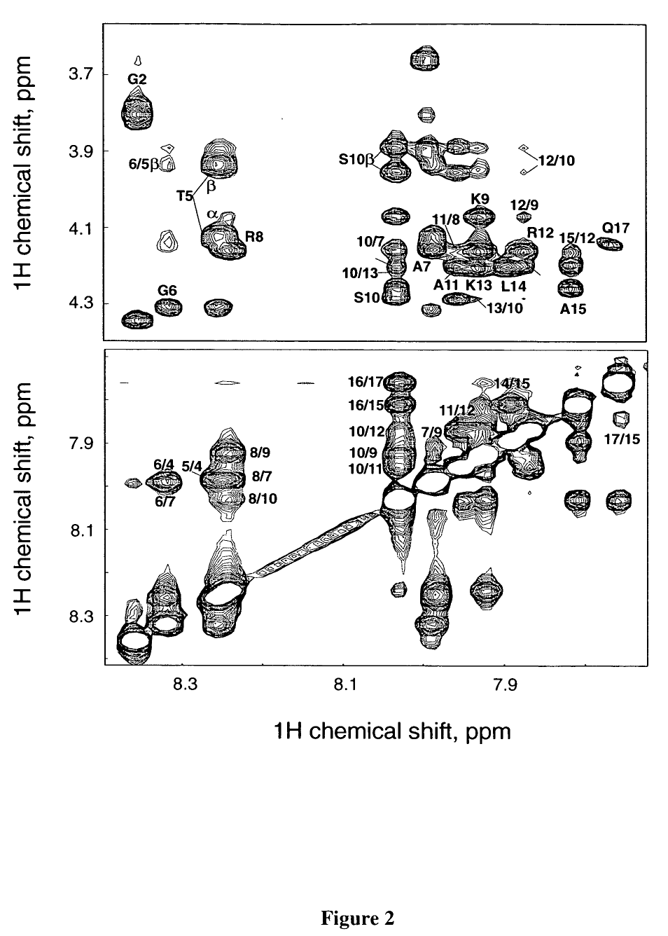

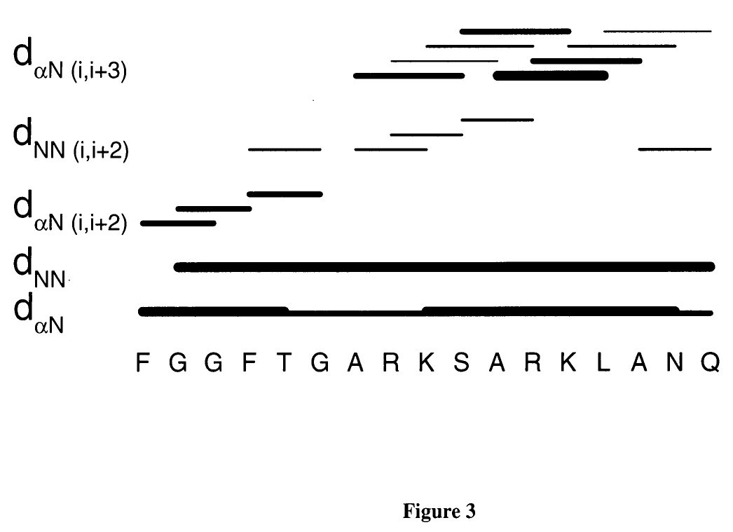

FIG. 2 shows the αH—NH and NH—NH regions from NOESY data on nociceptin in the presence of SDS micelles, and FIG. 3 summarizes these and other observed NOEs. The pattern of NOEs indicates a relatively well-formed helix conformation (Wüthrich et al., NMR of Proteins and Nucleic Acids, Wiley-Interscience, NY (1986)).

Because nociceptin in a helix conformation is amphipathic and therefore could self-associate, thereby affecting NOE data, PFG NMR diffusion measurements were made to check for aggregation. Diffusion coefficients derived from these data remain unchanged over the peptide concentration range of 0.1 mM to 15 mM, indicating the absence of aggregation (data not shown). Moreover, as a function of peptide concentration, the nociceptin diffusion coefficient varied linearly in a fashion expected for increased viscosity from a monomeric peptide the size of nociceptin.

Conformational Modeling. Conformational modeling was performed using NOE data acquired for the peptide in the presence of SDS micelles. A total of 140 NOE distance constraints were derived from analysis of NOESY spectra. These include 68 intraresidue, 24 sequential, 20 medium-range (|i-j|<5), and 28 long-range (|i-j|≧5) constraints. In addition, a total of eigth hydrogen bonds could be identified by inspection of initial structures and from long lived backbone NHs, giving rise to 16 hydrogen bond distance constraints. The total number of experimentally derived constraints was therefore 156, giving an average of 13 constraints per residue.

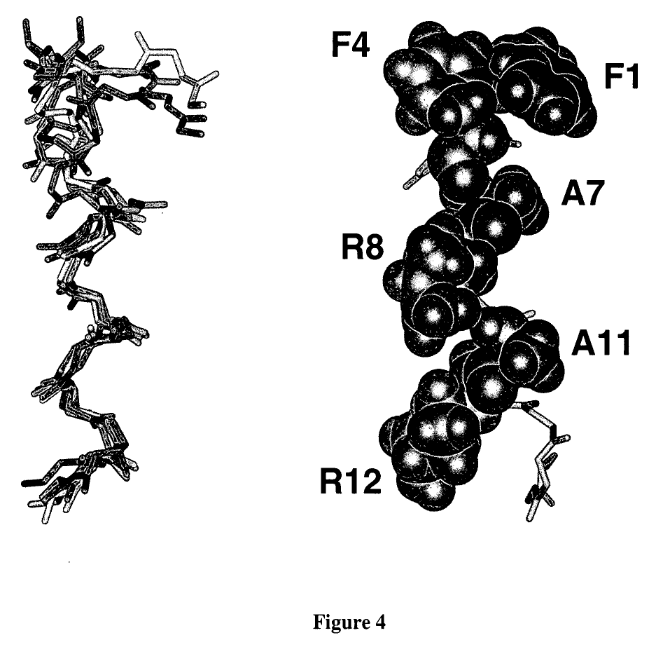

Initially, 100 structures for nociceptin were calculated using the methods described in the Methods Section. The best fit superpositions of backbone Cα atoms for the final 14 structures are shown in FIG. 4A. The parameters used for this determination were: total energy of less than 100 kcal/mol; and NOE violations no greater than 0.5 Angstrom. These structures showed no NOE violations greater than 0.5 Å. Structural statistics are summarized in Table 2.

TABLE 2
Structural Statistics for the calculated structures of
nociceptin from NMR data.
RMS Deviations from experimental distance restraints (Å)^{a}

| | |
|---|---|
| NOE (140) | 0.074 ± 0.02 |
| H-bond (16) | |

Deviations from idealized geometry

| | |
|---|---|
| Bonds (Å) | 0.0037 ± 0.00065 |
| Angles (°) | 0.63 ± 0.05 |

Energies (kcal · mol⁻¹)

| | |
|---|---|
| ENOE^{b} | 33 ± 14.6 |
| EBOND | 3.6 ± 1.2 |
| EANGLE | 28 ± 4.2 |
| ETOTAL | 86 ± 32 |

^{a}None of the 14 final structures exhibited distance restraint violations greater than 0.5 Å or dihedral angle violations greater than 5°. RMSD values represent the mean and standard deviations for the 14 structures.
^{b}The final values of the NOE (ENOE), torsion angle (ECDIH) and NCS (ENCS) potentials have been calculated with force constants of 50 kcal · mol⁻¹ · Å⁻², 200 kcal · mol⁻¹ · rad⁻², and 300 kcal · mol⁻¹ · Å⁻², respectively.

The somewhat less structurally-defined N-terminus is apparent. These structures satisfy experimental constraints quite well. For residues 5 through 15, atomic RMS differences with respect to the mean coordinate positions are 0.71±0.08 Å for backbone (N,C^{α}, C) atoms and 1.4±0.4 Å for all heavy atoms. In addition, φ and ψ angular order parameters are all greater than (>) 0.8. Together, the above data indicate that these structures used to represent the solution conformation of nociceptin are well converged. The surface of one face of the helix is highly positively charged with two pairs of RK residues lying below the functionally important N-terminal phenylalanines, as illustrated in FIG. 4B.

3D Structures. A three-dimensional structure of nociceptin is represented by the Cartesian coordinates is listed in Table 4. In certain embodiments, a three-dimensional structure of a nociceptin pharmacophore is represented by the Cartesian coordinates listed in Table 5, and alternatively in Table 6. Each table can be saved as a text file with a .mol2 extension and read within a Windows operating system using WebLab Viewer Lite, version 4.0, available from Accelrys Inc., San Diego, Calif.

TABLE 4
@<TRIPOS>MOLECULE
NMR-nociceptin-phe1
262 263 17 3 1
PROTEIN
NO_CHARGES
@<TRIPOS>DICT
PROTEIN PROTEIN
@<TRIPOS>ATOM

| | | | | | | | | | |
|---|---|---|---|---|---|---|---|---|---|
| 1 | CA | 23.2150 | −2.2410 | 8.3810 | C.3 | 1 | PHE1 | 0.0000 | BACKBONE\|DICT\|DIRECT |
| 2 | HA | 24.0680 | −1.6320 | 8.1280 | H | 1 | PHE1 | 0.0000 | BACKBONE\|DICT\|DIRECT |
| 3 | CB | 23.2650 | −3.5580 | 7.6050 | C.3 | 1 | PHE1 | 0.0000 | DICT |
| 4 | HB1 | 22.8830 | −3.4020 | 6.6070 | H | 1 | PHE1 | 0.0000 | DICT |
| 5 | HB2 | 22.6610 | −4.2960 | 8.1110 | H | 1 | PHE1 | 0.0000 | DICT |
| 6 | CG | 24.6920 | −4.0430 | 7.5260 | C.ar | 1 | PHE1 | 0.0000 | DICT |
| 7 | CD1 | 25.0490 | −5.2650 | 8.1080 | C.ar | 1 | PHE1 | 0.0000 | DICT |
| 8 | HD1 | 24.3030 | −5.8600 | 8.6140 | H | 1 | PHE1 | 0.0000 | DICT |
| 9 | CD2 | 25.6590 | −3.2710 | 6.8700 | C.ar | 1 | PHE1 | 0.0000 | DICT |
| 10 | HD2 | 25.3840 | −2.3280 | 6.4210 | H | 1 | PHE1 | 0.0000 | DICT |
| 11 | CE1 | 26.3720 | −5.7160 | 8.0340 | C.ar | 1 | PHE1 | 0.0000 | DICT |
| 12 | HE1 | 26.6470 | −6.6590 | 8.4830 | H | 1 | PHE1 | 0.0000 | DICT |
| 13 | CE2 | 26.9820 | −3.7220 | 6.7960 | C.ar | 1 | PHE1 | 0.0000 | DICT |
| 14 | HE2 | 27.7280 | −3.1270 | 6.2900 | H | 1 | PHE1 | 0.0000 | DICT |
| 15 | CZ | 27.3390 | −4.9450 | 7.3780 | C.ar | 1 | PHE1 | 0.0000 | DICT |
| 16 | HZ | 28.3600 | −5.2930 | 7.3210 | H | 1 | PHE1 | 0.0000 | DICT |
| 17 | C | 21.9140 | −1.4920 | 8.0810 | C.2 | 1 | PHE1 | 0.0000 | BACKBONE\|DICT\|DIRECT |
| 18 | O | 21.9190 | −0.3140 | 7.7820 | O.2 | 1 | PHE1 | 0.0000 | BACKBONE\|DICT\|DIRECT |
| 19 | N | 23.2630 | −2.6360 | 9.8180 | N.am | 1 | PHE1 | 0.0000 | BACKBONE\|DICT\|DIRECT |
| 20 | HT1 | 24.0780 | −3.2620 | 9.9790 | H | 1 | PHE1 | 0.0000 | |
| 21 | HT2 | 22.3860 | −3.1370 | 10.0690 | H | 1 | PHE1 | 0.0000 | |
| 22 | HT3 | 23.3590 | −1.7870 | 10.4090 | H | 1 | PHE1 | 0.0000 | |
| 23 | N | 20.7990 | −2.1690 | 8.1570 | N.am | 2 | GLY2 | 0.0000 | BACKBONE\|DICT\|DIRECT |
| 24 | HN | 20.8200 | −3.1190 | 8.3990 | H | 2 | GLY2 | 0.0000 | |
| 25 | CA | 19.4970 | −1.5010 | 7.8770 | C.3 | 2 | GLY2 | 0.0000 | BACKBONE\|DICT\|DIRECT |
| 26 | HA1 | 19.6670 | −0.6110 | 7.2910 | H | 2 | GLY2 | 0.0000 | BACKBONE\|DICT\|DIRECT |
| 27 | HA2 | 19.0210 | −1.2350 | 8.8100 | H | 2 | GLY2 | 0.0000 | DICT |
| 28 | C | 18.5920 | −2.4550 | 7.0940 | C.2 | 2 | GLY2 | 0.0000 | BACKBONE\|DICT\|DIRECT |
| 29 | O | 18.6530 | −2.5290 | 5.8820 | O.2 | 2 | GLY2 | 0.0000 | BACKBONE\|DICT\|DIRECT |
| 30 | N | 17.7540 | −3.1850 | 7.7790 | N.am | 3 | GLY3 | 0.0000 | BACKBONE\|DICT\|DIRECT |
| 31 | HN | 17.7230 | −3.1080 | 8.7560 | H | 3 | GLY3 | 0.0000 | |
| 32 | CA | 16.8430 | −4.1360 | 7.0790 | C.3 | 3 | GLY3 | 0.0000 | BACKBONE\|DICT\|DIRECT |
| 33 | HA1 | 16.7410 | −5.0350 | 7.6670 | H | 3 | GLY3 | 0.0000 | BACKBONE\|DICT\|DIRECT |
| 34 | HA | 17.2570 | −4.3830 | 6.1120 | H | 3 | GLY3 | 0.0000 | DICT |
| 35 | C | 15.4650 | −3.4930 | 6.8960 | C. | 2 | GLY3 | 0.0000 | BACKBONE\|DICT\|DIRECT |
| 36 | O | 14.6820 | −3.9110 | 6.0660 | O.2 | 3 | GLY3 | 0.0000 | BACKBONE\|DICT\|DIRECT |
| 37 | N | 15.1620 | −2.4790 | 7.6650 | N.am | 4 | PHE4 | 0.0000 | BACKBONE\|DICT\|DIRECT |
| 38 | HN | 15.8080 | −2.1580 | 8.3290 | H | 4 | PHE4 | 0.0000 | |
| 39 | CA | 13.8330 | −1.8110 | 7.5350 | C.3 | 4 | PHE4 | 0.0000 | BACKBONE\|DICT\|DIRECT |
| 40 | HA | 13.0420 | −2.4910 | 7.8080 | H | 4 | PHE4 | 0.0000 | BACKBONE\|DICT\|DIRECT |
| 41 | CB | 13.8790 | −0.6410 | 8.5190 | C.3 | 4 | PHE4 | 0.0000 | DICT |
| 42 | HB1 | 13.2290 | 0.1480 | 8.1710 | H | 4 | PHE4 | 0.0000 | DICT |
| 43 | HB2 | 14.8910 | −0.2690 | 8.5890 | H | 4 | PHE4 | 0.0000 | DICT |
| 44 | CG | 13.4180 | −1.1060 | 9.8800 | C.ar | 4 | PHE4 | 0.0000 | DICT |
| 45 | CD1 | 12.3140 | −0.5000 | 10.4910 | C.ar | 4 | PHE4 | 0.0000 | DICT |
| 46 | HD1 | 11.7920 | 0.3010 | 9.9880 | H | 4 | PHE4 | 0.0000 | DICT |
| 47 | CD2 | 14.0960 | −2.1440 | 10.5320 | C.ar | 4 | PHE4 | 0.0000 | DICT |
| 48 | HD2 | 14.9480 | −2.6130 | 10.0620 | H | 4 | PHE4 | 0.0000 | DICT |
| 49 | CE1 | 11.8870 | −0.9300 | 11.7530 | C.ar | 4 | PHE4 | 0.0000 | DICT |
| 50 | HE1 | 11.0360 | −0.4620 | 12.2230 | H | 4 | PHE4 | 0.0000 | DICT |
| 51 | CE2 | 13.6690 | −2.5750 | 11.7940 | C.ar | 4 | PHE4 | 0.0000 | DICT |
| 52 | HE2 | 14.1910 | −3.3750 | 12.2970 | H | 4 | PHE4 | 0.0000 | DICT |
| 53 | CZ | 12.5640 | −1.9680 | 12.4040 | C.ar | 4 | PHE4 | 0.0000 | DICT |
| 54 | HZ | 12.2350 | −2.3000 | 13.3780 | H | 4 | PHE4 | 0.0000 | DICT |
| 55 | C | 13.6310 | −1.3020 | 6.1030 | C.2 | 4 | PHE4 | 0.0000 | BACKBONE\|DICT\|DIRECT |
| 56 | O | 12.8850 | −1.8740 | 5.3330 | O.2 | 4 | PHE4 | 0.0000 | BACKBONE\|DICT\|DIRECT |
| 57 | N | 14.2900 | −0.2330 | 5.7440 | N.am | 5 | THR5 | 0.0000 | BACKBONE\|DICT\|DIRECT |
| 58 | HN | 14.8850 | 0.2120 | 6.3830 | H | 5 | THR5 | 0.0000 | |
| 59 | CA | 14.1350 | 0.3140 | 4.3620 | C.3 | 5 | THR5 | 0.0000 | BACKBONE\|DICT\|DIRECT |
| 60 | HA | 13.0950 | 0.5110 | 4.1530 | H | 5 | THR5 | 0.0000 | BACKBONE\|DICT\|DIRECT |
| 61 | CB | 14.9240 | 1.6260 | 4.3550 | C.3 | 5 | THR5 | 0.0000 | DICT |
| 62 | HB | 14.8280 | 2.0960 | 3.3880 | H | 5 | THR5 | 0.0000 | DICT |
| 63 | OG1 | 16.2950 | 1.3560 | 4.6150 | O.3 | 5 | THR5 | 0.0000 | DICT |
| 64 | HG1 | 16.3900 | 1.1650 | 5.5510 | H | 5 | THR5 | 0.0000 | DICT\|ESSENTIAL |
| 65 | CG2 | 14.3700 | 2.5670 | 5.4280 | C.3 | 5 | THR5 | 0.0000 | DICT |
| 66 | HG21 | 14.7210 | 2.2510 | 6.3990 | H | 5 | THR5 | 0.0000 | DICT |
| 67 | HG22 | 13.2910 | 2.5400 | 5.4080 | H | 5 | THR5 | 0.0000 | DICT |
| 68 | HG23 | 14.7090 | 3.5740 | 5.2330 | H | 5 | THR5 | 0.0000 | DICT |
| 69 | C | 14.7100 | −0.6610 | 3.3290 | C.2 | 5 | THR5 | 0.0000 | BACKBONE\|DICT\|DIRECT |
| 70 | O | 14.4570 | −0.5430 | 2.1460 | O.2 | 5 | THR5 | 0.0000 | BACKBONE\|DICT\|DIRECT |
| 71 | N | 15.4860 | −1.6220 | 3.7630 | N.am | 6 | GLY6 | 0.0000 | BACKBONE\|DICT\|DIRECT |
| 72 | HN | 15.6810 | −1.7000 | 4.7200 | H | 6 | GLY6 | 0.0000 | |
| 73 | CA | 16.0790 | −2.6000 | 2.8030 | C.3 | 6 | GLY6 | 0.0000 | BACKBONE\|DICT\|DIRECT |
| 74 | HA1 | 16.6400 | −3.3440 | 3.3480 | H | 6 | GLY6 | 0.0000 | BACKBONE\|DICT\|DIRECT |
| 75 | HA2 | 16.7370 | −2.0790 | 2.1220 | H | 6 | GLY6 | 0.0000 | DICT |
| 76 | C | 14.9660 | −3.2900 | 2.0080 | C.2 | 6 | GLY6 | 0.0000 | BACKBONE\|DICT\|DIRECT |
| 77 | O | 15.0330 | −3.4000 | 0.7990 | O.2 | 6 | GLY6 | 0.0000 | BACKBONE\|DICT\|DIRECT |
| 78 | N | 13.9450 | −3.7550 | 2.6770 | N.am | 7 | ALA7 | 0.0000 | BACKBONE\|DICT\|DIRECT |
| 79 | HN | 13.9110 | −3.6560 | 3.6510 | H | 7 | ALA7 | 0.0000 | |
| 80 | CA | 12.8300 | −4.4380 | 1.9570 | C.3 | 7 | ALA7 | 0.0000 | BACKBONE\|DICT\|DIRECT |
| 81 | HA | 13.1190 | −4.6660 | 0.9440 | H | 7 | ALA7 | 0.0000 | BACKBONE\|DICT\|DIRECT |
| 82 | CB | 12.5900 | −5.7330 | 2.7340 | C.3 | 7 | ALA7 | 0.0000 | DICT |
| 83 | HB1 | 12.9940 | −5.6350 | 3.7310 | H | 7 | ALA7 | 0.0000 | DICT |
| 84 | HB2 | 13.0770 | −6.5530 | 2.2270 | H | 7 | ALA7 | 0.0000 | DICT |
| 85 | HB3 | 11.5290 | −5.9260 | 2.7940 | H | 7 | ALA7 | 0.0000 | DICT |
| 86 | C | 11.5730 | −3.5630 | 1.9710 | C.2 | 7 | ALA7 | 0.0000 | BACKBONE\|DICT\|DIRECT |
| 87 | O | 10.7660 | −3.6070 | 1.0640 | O.2 | 7 | ALA7 | 0.0000 | BACKBONE\|DICT\|DIRECT |
| 88 | N | 11.4040 | −2.7690 | 2.9940 | N.am | 8 | ARG8 | 0.0000 | BACKBONE\|DICT\|DIRECT |
| 89 | HN | 12.0680 | −2.7510 | 3.7150 | H | 8 | ARG8 | 0.0000 | |
| 90 | CA | 10.1990 | −1.8900 | 3.0680 | C.3 | 8 | ARG8 | 0.0000 | BACKBONE\|DICT\|DIRECT |
| 91 | HA | 9.3030 | −2.4630 | 2.8950 | H | 8 | ARG8 | 0.0000 | BACKBONE\|DICT\|DIRECT |
| 92 | CB | 10.1970 | −1.3370 | 4.4950 | C.3 | 8 | ARG8 | 0.0000 | DICT |
| 93 | HB1 | 10.7380 | −0.4020 | 4.5190 | H | 8 | ARG8 | 0.0000 | DICT |
| 94 | HB2 | 10.6740 | −2.0460 | 5.1550 | H | 8 | ARG8 | 0.0000 | DICT |
| 95 | CG | 8.7560 | −1.1020 | 4.9530 | C.3 | 8 | ARG8 | 0.0000 | DICT |
| 96 | HG1 | 8.6610 | −1.3730 | 5.9940 | H | 8 | ARG8 | 0.0000 | DICT |
| 97 | HG2 | 8.0870 | −1.7090 | 4.3600 | H | 8 | ARG8 | 0.0000 | DICT |
| 98 | CD | 8.3960 | 0.3750 | 4.7780 | C.3 | 8 | ARG8 | 0.0000 | DICT |
| 99 | HD1 | 8.6840 | 0.7200 | 3.7980 | H | 8 | ARG8 | 0.0000 | DICT |
| 100 | HD2 | 8.8750 | 0.9700 | 5.5440 | H | 8 | ARG8 | 0.0000 | DICT |
| 101 | NE | 6.9150 | 0.4290 | 4.9240 | N.pl3 | 8 | ARG8 | 0.0000 | DICT |
| 102 | HE | 6.3490 | 0.5480 | 4.1330 | H | 8 | ARG8 | 0.0000 | DICT\|ESSENTIAL |
| 103 | CZ | 6.3710 | 0.3180 | 6.1050 | C.cat | 8 | ARG8 | 0.0000 | DICT |
| 104 | NH1 | 6.8590 | 0.9860 | 7.1150 | N.pl3 | 8 | ARG8 | 0.0000 | DICT |
| 105 | HH11 | 7.6500 | 1.5830 | 6.9830 | H | 8 | ARG8 | 0.0000 | DICT\|ESSENTIAL |
| 106 | HH12 | 6.4420 | 0.9000 | 8.0200 | H | 8 | ARG8 | 0.0000 | DICT\|ESSENTIAL |
| 107 | NH2 | 5.3380 | −0.4610 | 6.2770 | N.pl3 | 8 | ARG8 | 0.0000 | DICT |
| 108 | HH21 | 4.9630 | −0.9730 | 5.5040 | H | 8 | ARG8 | 0.0000 | DICT\|ESSENTIAL |
| 109 | HH22 | 4.9210 | −0.5460 | 7.1820 | H | 8 | ARG8 | 0.0000 | DICT\|ESSENTIAL |
| 110 | C | 10.3130 | −0.7510 | 2.0510 | C.2 | 8 | ARG8 | 0.0000 | BACKBONE\|DICT\|DIRECT |
| 111 | O | 9.3360 | −0.3380 | 1.4570 | O.2 | 8 | ARG8 | 0.0000 | BACKBONE\|DICT\|DIRECT |
| 112 | N | 11.4980 | −0.2420 | 1.8470 | N.am | 9 | LYS9 | 0.0000 | BACKBONE\|DICT\|DIRECT |
| 113 | HN | 12.2710 | −0.5920 | 2.3390 | H | 9 | LYS9 | 0.0000 | |
| 114 | CA | 11.6790 | 0.8710 | 0.8700 | C.3 | 9 | LYS9 | 0.0000 | BACKBONE\|DICT\|DIRECT |
| 115 | HA | 10.8330 | 1.5390 | 0.8990 | H | 9 | LYS9 | 0.0000 | BACKBONE\|DICT\|DIRECT |
| 116 | CB | 12.9380 | 1.6030 | 1.3340 | C.3 | 9 | LYS9 | 0.0000 | DICT |
| 117 | HB1 | 13.7940 | 1.2280 | 0.7930 | H | 9 | LYS9 | 0.0000 | DICT |
| 118 | HB2 | 13.0800 | 1.4380 | 2.3930 | H | 9 | LYS9 | 0.0000 | DICT |
| 119 | CG | 12.7860 | 3.1020 | 1.0660 | C.3 | 9 | LYS9 | 0.0000 | DICT |
| 120 | HG1 | 11.7720 | 3.4050 | 1.2780 | H | 9 | LYS9 | 0.0000 | DICT |
| 121 | HG2 | 13.0150 | 3.3070 | 0.0300 | H | 9 | LYS9 | 0.0000 | DICT |
| 122 | CD | 13.7470 | 3.8810 | 1.9660 | C.3 | 9 | LYS9 | 0.0000 | DICT |
| 123 | HD1 | 14.7630 | 3.5890 | 1.7470 | H | 9 | LYS9 | 0.0000 | DICT |
| 124 | HD2 | 13.5250 | 3.6650 | 3.0020 | H | 9 | LYS9 | 0.0000 | DICT |
| 125 | CE | 13.5840 | 5.3810 | 1.7110 | C.3 | 9 | LYS9 | 0.0000 | DICT |
| 126 | HE1 | 12.5960 | 5.5940 | 1.3330 | H | 9 | LYS9 | 0.0000 | DICT |
| 127 | HE2 | 14.3380 | 5.7270 | 1.0180 | H | 9 | LYS9 | 0.0000 | DICT |
| 128 | NZ | 13.7680 | 6.0190 | 3.0450 | N.4 | 9 | LYS9 | 0.0000 | DICT |
| 129 | HZ1 | 14.7300 | 5.8310 | 3.3900 | H | 9 | LYS9 | 0.0000 | DICT\|ESSENTIAL |
| 130 | HZ2 | 13.0760 | 5.6260 | 3.7150 | H | 9 | LYS9 | 0.0000 | DICT\|ESSENTIAL |
| 131 | HZ3 | 13.6270 | 7.0460 | 2.9610 | H | 9 | LYS9 | 0.0000 | DICT\|ESSENTIAL |
| 132 | C | 11.8740 | 0.3150 | −0.5460 | C.2 | 9 | LYS9 | 0.0000 | BACKBONE\|DICT\|DIRECT |
| 133 | O | 11.6790 | 1.0100 | −1.5240 | O.2 | 9 | LYS9 | 0.0000 | BACKBONE\|DICT\|DIRECT |
| 134 | N | 12.2580 | −0.9300 | −0.6630 | N.am | 10 | SER10 | 0.0000 | BACKBONE\|DICT\|DIRECT |
| 135 | HN | 12.4110 | −1.4730 | 0.1380 | H | 10 | SER10 | 0.0000 | |
| 136 | CA | 12.4660 | −1.5250 | −2.0180 | C.3 | 10 | SER10 | 0.0000 | BACKBONE\|DICT\|DIRECT |
| 137 | HA | 13.2880 | −1.0400 | −2.5200 | H | 10 | SER10 | 0.0000 | BACKBONE\|DICT\|DIRECT |
| 138 | CB | 12.8060 | −2.9930 | −1.7620 | C.3 | 10 | SER10 | 0.0000 | DICT |
| 139 | HB1 | 12.3280 | −3.6080 | −2.5130 | H | 10 | SER10 | 0.0000 | DICT |
| 140 | HB2 | 12.4510 | −3.2820 | −0.7860 | H | 10 | SER10 | 0.0000 | DICT |
| 141 | OG | 14.2160 | −3.1640 | −1.8170 | O.3 | 10 | SER10 | 0.0000 | DICT |
| 142 | HG | 14.3950 | −4.0890 | −2.0010 | H | 10 | SER10 | 0.0000 | DICT\|ESSENTIAL |
| 143 | C | 11.1870 | −1.4090 | −2.8540 | C.2 | 10 | SER10 | 0.0000 | BACKBONE\|DICT\|DIRECT |
| 144 | 0 | 11.2310 | −1.3620 | −4.0680 | O.2 | 10 | SER10 | 0.0000 | BACKBONE\|DICT\|DIRECT |
| 145 | N | 10.0490 | −1.3620 | −2.2140 | N.am | 11 | ALA11 | 0.0000 | BACKBONE\|DICT\|DIRECT |
| 146 | HN | 10.0370 | −1.4020 | −1.2340 | H | 11 | ALA11 | 0.0000 | |
| 147 | CA | 8.7680 | −1.2490 | −2.9720 | C.3 | 11 | ALA11 | 0.0000 | BACKBONE\|DICT\|DIRECT |
| 148 | HA | 8.6480 | −2.0900 | −3.6360 | H | 11 | ALA11 | 0.0000 | BACKBONE\|DICT\|DIRECT |
| 149 | CB | 7.6700 | −1.2630 | −1.9080 | C.3 | 11 | ALA11 | 0.0000 | DICT |
| 150 | HB1 | 7.9870 | −0.6800 | −1.0550 | H | 11 | ALA11 | 0.0000 | DICT |
| 151 | HB2 | 7.4810 | −2.2800 | −1.5980 | H | 11 | ALA11 | 0.0000 | DICT |
| 152 | HB3 | 6.7650 | −0.8380 | −2.3180 | H | 11 | ALA11 | 0.0000 | DICT |
| 153 | C | 8.7360 | 0.0630 | −3.7610 | C.2 | 11 | ALA11 | 0.0000 | BACKBONE\|DICT\|DIRECT |
| 154 | O | 8.4220 | 0.0840 | −4.9350 | O.2 | 11 | ALA11 | 0.0000 | BACKBONE\|DICT\|DIRECT |
| 155 | N | 9.0620 | 1.1570 | −3.1250 | N.am | 12 | ARG12 | 0.0000 | BACKBONE\|DICT\|DIRECT |
| 156 | HN | 9.3130 | 1.1160 | −2.1780 | H | 12 | ARG12 | 0.0000 | |
| 157 | CA | 9.0530 | 2.4680 | −3.8370 | C.3 | 12 | ARG12 | 0.0000 | BACKBONE\|DICT\|DIRECT |
| 158 | HA | 8.3020 | 2.4730 | −4.6100 | H | 12 | ARG12 | 0.0000 | BACKBONE\|DICT\|DIRECT |
| 159 | CB | 8.7070 | 3.5020 | −2.7650 | C.3 | 12 | ARG12 | 0.0000 | DICT |
| 160 | HB1 | 8.7340 | 4.4910 | −3.1960 | H | 12 | ARG12 | 0.0000 | DICT |
| 161 | HB2 | 9.4260 | 3.4400 | −1.9600 | H | 12 | ARG12 | 0.0000 | DICT |
| 162 | CG | 7.3050 | 3.2230 | −2.2200 | C.3 | 12 | ARG12 | 0.0000 | DICT |
| 163 | HG1 | 7.3340 | 2.3570 | −1.5770 | H | 12 | ARG12 | 0.0000 | DICT |
| 164 | HG2 | 6.6300 | 3.0380 | −3.0430 | H | 12 | ARG12 | 0.0000 | DICT |
| 165 | CD | 6.8140 | 4.4340 | −1.4190 | C.3 | 12 | ARG12 | 0.0000 | DICT |
| 166 | HD1 | 5.8810 | 4.7980 | −1.8200 | H | 12 | ARG12 | 0.0000 | DICT |
| 167 | HD2 | 7.5600 | 5.2180 | −1.4270 | H | 12 | ARG12 | 0.0000 | DICT |
| 168 | NE | 6.6100 | 3.9240 | −0.0350 | N.p13 | 12 | ARG12 | 0.0000 | DICT |
| 169 | HE | 7.3830 | 3.6930 | 0.5210 | H | 12 | ARG12 | 0.0000 | DICT\|ESSENTIAL |
| 170 | CZ | 5.4010 | 3.7830 | 0.4370 | C.cat | 12 | ARG12 | 0.0000 | DICT |
| 171 | NH1 | 4.8680 | 2.5950 | 0.5200 | N.p13 | 12 | ARG12 | 0.0000 | DICT |
| 172 | HH11 | 5.3850 | 1.7930 | 0.2230 | H | 12 | ARG12 | 0.0000 | DICT\|ESSENTIAL |
| 173 | HH12 | 3.9420 | 2.4870 | 0.8820 | H | 12 | ARG12 | 0.0000 | DICT\|ESSENTIAL |
| 174 | NH2 | 4.7270 | 4.8310 | 0.8250 | N.p13 | 12 | ARG12 | 0.0000 | DICT |
| 175 | HH21 | 5.1360 | 5.7410 | 0.7600 | H | 12 | ARG12 | 0.0000 | DICT\|ESSENTIAL |
| 176 | HH22 | 3.8010 | 4.7230 | 1.1860 | H | 12 | ARG12 | 0.0000 | DICT\|ESSENTIAL |
| 177 | C | 10.4350 | 2.7520 | −4.4310 | C.2 | 12 | ARG12 | 0.0000 | BACKBONE\|DICT\|DIRECT |
| 178 | O | 10.5630 | 3.4250 | −5.4350 | O.2 | 12 | ARG12 | 0.0000 | BACKBONE\|DICT\|DIRECT |
| 179 | N | 11.4700 | 2.2440 | −3.8170 | N.am | 13 | LYS13 | 0.0000 | BACKBONE\|DICT\|DIRECT |
| 180 | HN | 11.3420 | 1.7050 | −3.0080 | H | 13 | LYS13 | 0.0000 | |
| 181 | CA | 12.8450 | 2.4830 | −4.3440 | C.3 | 13 | LYS13 | 0.0000 | BACKBONE\|DICT\|DIRECT |
| 182 | HA | 13.0640 | 3.5390 | −4.3620 | H | 13 | LYS13 | 0.0000 | BACKBONE\|DICT\|DIRECT |
| 183 | CB | 13.7770 | 1.7730 | −3.3620 | C.3 | 13 | LYS13 | 0.0000 | DICT |
| 184 | HB1 | 13.6610 | 0.7040 | −3.4640 | H | 13 | LYS13 | 0.0000 | DICT |
| 185 | HB2 | 13.5290 | 2.0680 | −2.3530 | H | 13 | LYS13 | 0.0000 | DICT |
| 186 | CG | 15.2270 | 2.1560 | −3.6640 | C.3 | 13 | LYS13 | 0.0000 | DICT |
| 187 | HG1 | 15.2580 | 3.1560 | −4.0700 | H | 13 | LYS13 | 0.0000 | DICT |
| 188 | HG2 | 15.6410 | 1.4620 | −4.3820 | H | 13 | LYS13 | 0.0000 | DICT |
| 189 | CD | 16.0480 | 2.1060 | −2.3740 | C.3 | 13 | LYS13 | 0.0000 | DICT |
| 190 | HD1 | 15.5770 | 2.7230 | −1.6240 | H | 13 | LYS13 | 0.0000 | DICT |
| 191 | HD2 | 17.0450 | 2.4730 | −2.5680 | H | 13 | LYS13 | 0.0000 | DICT |
| 192 | CE | 16.1220 | 0.6630 | −1.8710 | C.3 | 13 | LYS13 | 0.0000 | DICT |
| 193 | HE1 | 16.3690 | −0.0050 | −2.6810 | H | 13 | LYS13 | 0.0000 | DICT |
| 194 | HE2 | 15.1840 | 0.3760 | −1.4160 | H | 13 | LYS13 | 0.0000 | DICT |
| 195 | NZ | 17.2140 | 0.6640 | −0.8590 | N.4 | 13 | LYS13 | 0.0000 | DICT |
| 196 | HZ1 | 17.2420 | −0.2560 | −0.3770 | H | 13 | LYS13 | 0.0000 | DICT\|ESSENTIAL |
| 197 | HZ2 | 17.0390 | 1.4170 | −0.1620 | H | 13 | LYS13 | 0.0000 | DICT\|ESSENTIAL |
| 198 | HZ3 | 18.1250 | 0.8330 | −1.3310 | H | 13 | LYS13 | 0.0000 | DICT\|ESSENTIAL |
| 199 | C | 12.9840 | 1.8840 | −5.7460 | C.2 | 13 | LYS13 | 0.0000 | BACKBONE\|DICT\|DIRECT |
| 200 | O | 13.7600 | 2.3500 | −6.5570 | O.2 | 13 | LYS13 | 0.0000 | BACKBONE\|DICT\|DIRECT |
| 201 | N | 12.2350 | 0.8530 | −6.0360 | N.am | 14 | LEU14 | 0.0000 | BACKBONE\|DICT\|DIRECT |
| 202 | HN | 11.6160 | 0.4940 | −5.3670 | H | 14 | LEU14 | 0.0000 | |
| 203 | CA | 12.3210 | 0.2220 | −7.3850 | C.3 | 14 | LEU14 | 0.0000 | BACKBONE\|DICT\|DIRECT |
| 204 | HA | 13.3220 | 0.3040 | −7.7770 | H | 14 | LEU14 | 0.0000 | BACKBONE\|DICT\|DIRECT |
| 205 | CB | 11.9690 | −1.2480 | −7.1560 | C.3 | 14 | LEU14 | 0.0000 | DICT |
| 206 | HB1 | 11.7630 | −1.7210 | −8.1040 | H | 14 | LEU14 | 0.0000 | DICT |
| 207 | HB2 | 11.0960 | −1.3140 | −6.5220 | H | 14 | LEU14 | 0.0000 | DICT |
| 208 | CG | 13.1450 | −1.9560 | −6.4810 | C.3 | 14 | LEU14 | 0.0000 | DICT |
| 209 | HG | 13.5270 | −1.3360 | −5.6820 | H | 14 | LEU14 | 0.0000 | DICT |
| 210 | CD1 | 12.6730 | −3.2930 | −5.9060 | C.3 | 14 | LEU14 | 0.0000 | DICT |
| 211 | HD11 | 12.8320 | −4.0740 | −6.6350 | H | 14 | LEU14 | 0.0000 | DICT |
| 212 | HD12 | 11.6220 | −3.2310 | −5.6680 | H | 14 | LEU14 | 0.0000 | DICT |
| 213 | HD13 | 13.2330 | −3.5170 | −5.0100 | H | 14 | LEU14 | 0.0000 | DICT |
| 214 | CD2 | 14.2480 | −2.2050 | −7.5110 | C.3 | 14 | LEU14 | 0.0000 | DICT |
| 215 | HD21 | 14.9940 | −2.8620 | −7.0880 | H | 14 | LEU14 | 0.0000 | DICT |
| 216 | HD22 | 14.7080 | −1.2660 | −7.7800 | H | 14 | LEU14 | 0.0000 | DICT |
| 217 | HD23 | 13.8220 | −2.6630 | −8.3910 | H | 14 | LEU14 | 0.0000 | DICT |
| 218 | C | 11.3150 | 0.8730 | −8.3380 | C.2 | 14 | LEU14 | 0.0000 | BACKBONE\|DICT\|DIRECT |
| 219 | O | 11.5830 | 1.0510 | −9.5110 | O.2 | 14 | LEU14 | 0.0000 | BACKBONE\|DICT\|DIRECT |
| 220 | N | 10.1600 | 1.2300 | −7.8430 | N.am | 15 | ALA15 | 0.0000 | BACKBONE\|DICT\|DIRECT |
| 221 | HN | 9.9670 | 1.0770 | −6.8950 | H | 15 | ALA15 | 0.0000 | |
| 222 | CA | 9.1350 | 1.8700 | −8.7180 | C.3 | 15 | ALA15 | 0.0000 | BACKBONE\|DICT\|DIRECT |
| 223 | HA | 9.1500 | 1.4290 | −9.7020 | H | 15 | ALA15 | 0.0000 | BACKBONE\|DICT\|DIRECT |
| 224 | CB | 7.7980 | 1.5830 | −8.0360 | C.3 | 15 | ALA15 | 0.0000 | DICT |
| 225 | HB1 | 7.7260 | 2.1600 | −7.1260 | H | 15 | ALA15 | 0.0000 | DICT |
| 226 | HB2 | 7.7320 | 0.5310 | −7.8010 | H | 15 | ALA15 | 0.0000 | DICT |
| 227 | HB3 | 6.9890 | 1.8550 | −8.6990 | H | 15 | ALA15 | 0.0000 | DICT |
| 228 | C | 9.3820 | 3.3790 | −8.8050 | C.2 | 15 | ALA15 | 0.0000 | BACKBONE\|DICT\|DIRECT |
| 229 | O | 9.4940 | 3.9390 | −9.8780 | O.2 | 15 | ALA15 | 0.0000 | BACKBONE\|DICT\|DIRECT |
| 230 | N | 9.4670 | 4.0410 | −7.6810 | N.am | 16 | GLU16 | 0.0000 | BACKBONE\|DICT\|DIRECT |
| 231 | HN | 9.3730 | 3.5670 | −6.8280 | H | 16 | GLU16 | 0.0000 | |
| 232 | CA | 9.7060 | 5.5140 | −7.6940 | C.3 | 16 | GLU16 | 0.0000 | BACKBONE\|DICT\|DIRECT |
| 233 | HA | 8.9050 | 6.0220 | −8.2080 | H | 16 | GLU16 | 0.0000 | BACKBONE\|DICT\|DIRECT |
| 234 | CB | 9.7270 | 5.9270 | −6.2220 | C.3 | 16 | GLU16 | 0.0000 | DICT |
| 235 | HB1 | 10.7490 | 5.9910 | −5.8800 | H | 16 | GLU16 | 0.0000 | DICT |
| 236 | HB2 | 9.1960 | 5.1920 | −5.6340 | H | 16 | GLU16 | 0.0000 | DICT |
| 237 | CG | 9.0530 | 7.2910 | −6.0630 | C.3 | 16 | GLU16 | 0.0000 | DICT |
| 238 | HG1 | 8.6510 | 7.3810 | −5.0650 | H | 16 | GLU16 | 0.0000 | DICT |
| 239 | HG2 | 8.2540 | 7.3840 | −6.7840 | H | 16 | GLU16 | 0.0000 | DICT |
| 240 | CD | 10.0820 | 8.4000 | −6.2960 | C.2 | 16 | GLU16 | 0.0000 | DICT |
| 241 | OE1 | 10.6920 | 8.4020 | −7.3520 | O.co2 | 16 | GLU16 | 0.0000 | DICT |
| 242 | OE2 | 10.2420 | 9.2270 | −5.4130 | O.co2 | 16 | GLU16 | 0.0000 | DICT |
| 243 | C | 11.0500 | 5.8270 | −8.3580 | C.2 | 16 | GLU16 | 0.0000 | BACKBONE\|DICT\|DIRECT |
| 244 | O | 11.1590 | 6.7290 | −9.1660 | O.2 | 16 | GLU16 | 0.0000 | BACKBONE\|DICT\|DIRECT |
| 245 | N | 12.0740 | 5.0880 | −8.0210 | N.am | 17 | GLN17 | 0.0000 | BACKBONE\|DICT\|DIRECT |
| 246 | HN | 11.9630 | 4.3670 | −7.3670 | H | 17 | GLN17 | 0.0000 | |
| 247 | CA | 13.4120 | 5.3420 | −8.6310 | C.3 | 17 | GLN17 | 0.0000 | BACKBONE\|DICT\|DIRECT |
| 248 | HA | 13.4920 | 6.3690 | −8.9490 | H | 17 | GLN17 | 0.0000 | BACKBONE\|DICT\|DIRECT |
| 249 | CB | 14.4190 | 5.0600 | −7.5120 | C.3 | 17 | GLN17 | 0.0000 | DICT |
| 250 | HB1 | 15.0320 | 4.2130 | −7.7830 | H | 17 | GLN17 | 0.0000 | DICT |
| 251 | HB2 | 13.8880 | 4.8420 | −6.5960 | H | 17 | GLN17 | 0.0000 | DICT |
| 252 | CG | 15.3120 | 62850 | −7.3040 | C.3 | 17 | GLN17 | 0.0000 | DICT |
| 253 | HG1 | 15.4790 | 6.7740 | −8.2510 | H | 17 | GLN17 | 0.0000 | DICT |
| 254 | HG2 | 16.2590 | 5.9730 | −6.8880 | H | 17 | GLN17 | 0.0000 | DICT |
| 255 | CD | 14.6270 | 7.2590 | −6.3430 | C.2 | 17 | GLN17 | 0.0000 | DICT |
| 256 | OE1 | 14.4220 | 8.4110 | −6.6720 | O.2 | 17 | GLN17 | 0.0000 | DICT |
| 257 | NE2 | 14.2630 | 6.8430 | −5.1610 | N.am | 17 | GLN17 | 0.0000 | DICT |
| 258 | HE21 | 14.4290 | 5.9150 | −4.8950 | H | 17 | GLN17 | 0.0000 | DICT\|ESSENTIAL |
| 259 | HE22 | 13.8240 | 7.4600 | −4.5380 | H | 17 | GLN17 | 0.0000 | DICT\|ESSENTIAL |
| 260 | C | 13.6410 | 4.3960 | −9.8130 | C.2 | 17 | GLN17 | 0.0000 | BACKBONE\|DICT\|DIRECT |
| 261 | OT1 | 13.0040 | 3.3560 | −9.8470 | O.2 | 17 | GLN17 | 0.0000 | |
| 262 | OT2 | 14.4500 | 4.7290 | −10.6640 | O.2 | 17 | GLN17 | 0.0000 | |

@<TRIPOS>BOND

| | | | | |
|---|---|---|---|---|
| 1 | 1 | 19 | 1 | BACKBONE\|DICT |
| 2 | 1 | 3 | 1 | DICT |
| 3 | 1 | 17 | 1 | BACKBONE\|DICT |
| 4 | 17 | 18 | 2 | BACKBONE\|DICT |
| 5 | 3 | 6 | 1 | DICT |
| 6 | 6 | 7 | ar | DICT |
| 7 | 6 | 9 | ar | DICT |
| 8 | 9 | 13 | ar | DICT |
| 9 | 13 | 15 | ar | DICT |
| 10 | 15 | 11 | ar | DICT |
| 11 | 11 | 7 | ar | DICT |
| 12 | 1 | 2 | 1 | BACKBONE\|DICT |
| 13 | 3 | 5 | 1 | DICT |
| 14 | 3 | 4 | 1 | DICT |
| 15 | 7 | 8 | 1 | DICT |
| 16 | 9 | 10 | 1 | DICT |
| 17 | 11 | 12 | 1 | DICT |
| 18 | 13 | 14 | 1 | DICT |
| 19 | 15 | 16 | 1 | DICT |
| 20 | 19 | 20 | 1 | |
| 21 | 19 | 21 | 1 | |
| 22 | 19 | 22 | 1 | |
| 23 | 25 | 23 | 1 | BACKBONE\|DICT |
| 24 | 25 | 28 | 1 | BACKBONE\|DICT |
| 25 | 28 | 29 | 2 | BACKBONE\|DICT |
| 26 | 25 | 26 | 1 | BACKBONE\|DICT |
| 27 | 25 | 27 | 1 | DICT |
| 28 | 23 | 24 | 1 | |
| 29 | 17 | 23 | am | BACKBONE\|DICT\|INTERRES |
| 30 | 32 | 30 | 1 | BACKBONE\|DICT |
| 31 | 32 | 35 | 1 | BACKBONE\|DICT |
| 32 | 35 | 36 | 2 | BACKBONE\|DICT |
| 33 | 32 | 33 | 1 | BACKBONE\|DICT |
| 34 | 32 | 34 | 1 | DICT |
| 35 | 30 | 31 | 1 | |
| 36 | 28 | 30 | am | BACKBONE\|DICT\|INTERRES |
| 37 | 39 | 37 | 1 | BACKBONE\|DICT |
| 38 | 39 | 41 | 1 | DICT |
| 39 | 39 | 55 | 1 | BACKBONE\|DICT |
| 40 | 55 | 56 | 2 | BACKBONE\|DICT |
| 41 | 41 | 44 | 1 | DICT |
| 42 | 44 | 45 | ar | DICT |
| 43 | 44 | 47 | ar | DICT |
| 44 | 47 | 51 | ar | DICT |
| 45 | 51 | 53 | ar | DICT |
| 46 | 53 | 49 | ar | DICT |
| 47 | 49 | 45 | ar | DICT |
| 48 | 39 | 40 | 1 | BACKBONE\|DICT |
| 49 | 41 | 43 | 1 | DICT |
| 50 | 41 | 42 | 1 | DICT |
| 51 | 45 | 46 | 1 | DICT |
| 52 | 47 | 48 | 1 | DICT |
| 53 | 49 | 50 | 1 | DICT |
| 54 | 51 | 52 | 1 | DICT |
| 55 | 53 | 54 | 1 | DICT |
| 56 | 37 | 38 | 1 | |
| 57 | 35 | 37 | am | BACKBONE\|DICT\|INTERRES |
| 58 | 59 | 57 | 1 | BACKBONE\|DICT |
| 59 | 59 | 61 | 1 | DICT |
| 60 | 59 | 69 | 1 | BACKBONE\|DICT |
| 61 | 69 | 70 | 2 | BACKBONE\|DICT |
| 62 | 61 | 65 | 1 | DICT |
| 63 | 61 | 63 | 1 | DICT |
| 64 | 59 | 60 | 1 | BACKBONE\|DICT |
| 65 | 61 | 62 | 1 | DICT |
| 66 | 65 | 67 | 1 | DICT |
| 67 | 65 | 66 | 1 | DICT |
| 68 | 65 | 68 | 1 | DICT |
| 69 | 63 | 64 | 1 | DICT |
| 70 | 57 | 58 | 1 | |
| 71 | 55 | 57 | am | BACKBONE\|DICT\|INTERRES |
| 72 | 73 | 71 | 1 | BACKBONE\|DICT |
| 73 | 73 | 76 | 1 | BACKBONE\|DICT |
| 74 | 76 | 77 | 2 | BACKBONE\|DICT |
| 75 | 73 | 74 | 1 | BACKBONE\|DICT |
| 76 | 73 | 75 | 1 | DICT |
| 77 | 71 | 72 | 1 | |
| 78 | 69 | 71 | am | BACKBONE\|DICT\|INTERRES |
| 79 | 80 | 78 | 1 | BACKBONE\|DICT |
| 80 | 80 | 82 | 1 | DICT |
| 81 | 80 | 86 | 1 | BACKBONE\|DICT |
| 82 | 86 | 87 | 2 | BACKBONE\|DICT |
| 83 | 80 | 81 | 1 | BACKBONE\|DICT |
| 84 | 82 | 85 | 1 | DICT |
| 85 | 82 | 84 | 1 | DICT |
| 86 | 82 | 83 | 1 | DICT |
| 87 | 78 | 79 | 1 | |
| 88 | 76 | 78 | am | BACKBONE\|DICT\|INTERRES |
| 89 | 90 | 88 | 1 | BACKBONE\|DICT |
| 90 | 90 | 92 | 1 | DICT |
| 91 | 90 | 110 | 1 | BACKBONE\|DICT |
| 92 | 110 | 111 | 2 | BACKBONE\|DICT |
| 93 | 92 | 95 | 1 | DICT |
| 94 | 95 | 98 | 1 | DICT |
| 95 | 98 | 101 | 1 | DICT |
| 96 | 101 | 103 | ar | DICT |
| 97 | 103 | 107 | ar | DICT |
| 98 | 103 | 104 | ar | DICT |
| 99 | 104 | 105 | 1 | DICT |
| 100 | 104 | 106 | 1 | DICT |
| 101 | 90 | 91 | 1 | BACKBONE\|DICT |
| 102 | 92 | 94 | 1 | DICT |
| 103 | 92 | 93 | 1 | DICT |
| 104 | 95 | 97 | 1 | DICT |
| 105 | 95 | 96 | 1 | DICT |
| 106 | 98 | 100 | 1 | DICT |
| 107 | 98 | 99 | 1 | DICT |
| 108 | 101 | 102 | 1 | DICT |
| 109 | 107 | 108 | 1 | DICT |
| 110 | 107 | 109 | 1 | DICT |
| 111 | 88 | 89 | 1 | |
| 112 | 86 | 88 | am | BACKBONE\|DICT\|INTERRES |
| 113 | 114 | 112 | 1 | BACKBONE\|DICT |
| 114 | 114 | 116 | 1 | DICT |
| 115 | 114 | 132 | 1 | BACKBONE\|DICT |
| 116 | 132 | 133 | 2 | BACKBONE\|DICT |
| 117 | 116 | 119 | 1 | DICT |
| 118 | 119 | 122 | 1 | DICT |
| 119 | 122 | 125 | 1 | DICT |
| 120 | 125 | 128 | 1 | DICT |
| 121 | 114 | 115 | 1 | BACKBONE\|DICT |
| 122 | 116 | 118 | 1 | DICT |
| 123 | 116 | 117 | 1 | DICT |
| 124 | 119 | 121 | 1 | DICT |
| 125 | 119 | 120 | 1 | DICT |
| 126 | 122 | 124 | 1 | DICT |
| 127 | 122 | 123 | 1 | DICT |
| 128 | 125 | 127 | 1 | DICT |
| 129 | 125 | 126 | 1 | DICT |
| 130 | 128 | 131 | 1 | DICT |
| 131 | 128 | 130 | 1 | DICT |
| 132 | 128 | 129 | 1 | DICT |
| 133 | 112 | 113 | 1 | |
| 134 | 110 | 112 | am | BACKBONE\|DICT\|INTERRES |
| 135 | 136 | 134 | 1 | BACKBONE\|DICT |
| 136 | 136 | 138 | 1 | DICT |
| 137 | 136 | 143 | 1 | BACKBONE\|DICT |
| 138 | 143 | 144 | 2 | BACKBONE\|DICT |
| 139 | 138 | 141 | 1 | DICT |
| 140 | 136 | 137 | 1 | BACKBONE\|DICT |
| 141 | 138 | 140 | 1 | DICT |
| 142 | 138 | 139 | 1 | DICT |
| 143 | 141 | 142 | 1 | DICT |
| 144 | 134 | 135 | 1 | |
| 145 | 132 | 134 | am | BACKBONE\|DICT\|INTERRES |
| 146 | 147 | 145 | 1 | BACKBONE\|DICT |
| 147 | 147 | 149 | 1 | DICT |
| 148 | 147 | 153 | 1 | BACKBONE\|DICT |
| 149 | 153 | 154 | 2 | BACKBONE\|DICT |
| 150 | 147 | 148 | 1 | BACKBONE\|DICT |
| 151 | 149 | 152 | 1 | DICT |
| 152 | 149 | 151 | 1 | DICT |
| 153 | 149 | 150 | 1 | DICT |
| 154 | 145 | 146 | 1 | |
| 155 | 143 | 145 | am | BACKBONE\|DICT\|INTERRES |
| 156 | 157 | 155 | 1 | BACKBONE\|DICT |
| 157 | 157 | 159 | 1 | DICT |
| 158 | 157 | 177 | 1 | BACKBONE\|DICT |
| 159 | 177 | 178 | 2 | BACKBONE\|DICT |
| 160 | 159 | 162 | 1 | DICT |
| 161 | 162 | 165 | 1 | DICT |
| 162 | 165 | 168 | 1 | DICT |
| 163 | 168 | 170 | ar | DICT |
| 164 | 170 | 174 | ar | DICT |
| 165 | 170 | 171 | ar | DICT |
| 166 | 171 | 172 | 1 | DICT |
| 167 | 171 | 173 | 1 | DICT |
| 168 | 157 | 158 | 1 | BACKBONE\|DICT |
| 169 | 159 | 161 | 1 | DICT |
| 170 | 159 | 160 | 1 | DICT |
| 171 | 162 | 164 | 1 | DICT |
| 172 | 162 | 163 | 1 | DICT |
| 173 | 165 | 167 | 1 | DICT |
| 174 | 165 | 166 | 1 | DICT |
| 175 | 168 | 169 | 1 | DICT |
| 176 | 174 | 175 | 1 | DICT |
| 177 | 174 | 176 | 1 | DICT |
| 178 | 155 | 156 | 1 | |
| 179 | 153 | 155 | am | BACKBONE\|DICT\|INTERRES |
| 180 | 181 | 179 | 1 | BACKBONE\|DICT |
| 181 | 181 | 183 | 1 | DICT |
| 182 | 181 | 199 | 1 | BACKBONE\|DICT |
| 183 | 199 | 200 | 2 | BACKBONE\|DICT |
| 184 | 183 | 186 | 1 | DICT |
| 185 | 186 | 189 | 1 | DICT |
| 186 | 189 | 192 | 1 | DICT |
| 187 | 192 | 195 | 1 | DICT |
| 188 | 181 | 182 | 1 | BACKBONE\|DICT |
| 189 | 183 | 185 | 1 | DICT |
| 190 | 183 | 184 | 1 | DICT |
| 191 | 186 | 188 | 1 | DICT |
| 192 | 186 | 187 | 1 | DICT |
| 193 | 189 | 191 | 1 | DICT |
| 194 | 189 | 190 | 1 | DICT |
| 195 | 192 | 194 | 1 | DICT |
| 196 | 192 | 193 | 1 | DICT |
| 197 | 195 | 198 | 1 | DICT |
| 198 | 195 | 197 | 1 | DICT |
| 199 | 195 | 196 | 1 | DICT |
| 200 | 179 | 180 | 1 | |
| 201 | 177 | 179 | am | BACKBONE\|DICT\|INTERRES |
| 202 | 203 | 201 | 1 | BACKBONE\|DICT |
| 203 | 203 | 205 | 1 | DICT |
| 204 | 203 | 218 | 1 | BACKBONE\|DICT |
| 205 | 218 | 219 | 2 | BACKBONE\|DICT |
| 206 | 205 | 208 | 1 | DICT |
| 207 | 208 | 210 | 1 | DICT |
| 208 | 208 | 214 | 1 | DICT |
| 209 | 203 | 204 | 1 | BACKBONE\|DICT |
| 210 | 205 | 207 | 1 | DICT |
| 211 | 205 | 206 | 1 | DICT |
| 212 | 208 | 209 | 1 | DICT |
| 213 | 210 | 213 | 1 | DICT |
| 214 | 210 | 212 | 1 | DICT |
| 215 | 210 | 211 | 1 | DICT |
| 216 | 214 | 217 | 1 | DICT |
| 217 | 214 | 216 | 1 | DICT |
| 218 | 214 | 215 | 1 | DICT |
| 219 | 201 | 202 | 1 | |
| 220 | 199 | 201 | am | BACKBONE\|DICT\|INTERRES |
| 221 | 222 | 220 | 1 | BACKBONE\|DICT |
| 222 | 222 | 224 | 1 | DICT |
| 223 | 222 | 228 | 1 | BACKBONE\|DICT |
| 224 | 228 | 229 | 2 | BACKBONE\|DICT |
| 225 | 222 | 223 | 1 | BACKBONE\|DICT |
| 226 | 224 | 227 | 1 | DICT |
| 227 | 224 | 226 | 1 | DICT |
| 228 | 224 | 225 | 1 | DICT |
| 229 | 220 | 221 | 1 | |
| 230 | 218 | 220 | am | BACKBONE\|DICT\|INTERRES |
| 231 | 232 | 230 | 1 | BACKBONE\|DICT |
| 232 | 232 | 234 | 1 | DICT |
| 233 | 232 | 243 | 1 | BACKBONE\|DICT |
| 234 | 243 | 244 | 2 | BACKBONE\|DICT |
| 235 | 234 | 237 | 1 | DICT |
| 236 | 237 | 240 | 1 | DICT |
| 237 | 240 | 242 | ar | DICT |
| 238 | 240 | 241 | ar | DICT |
| 239 | 232 | 233 | 1 | BACKBONE\|DICT |
| 240 | 234 | 236 | 1 | DICT |
| 241 | 234 | 235 | 1 | DICT |
| 242 | 237 | 239 | 1 | DICT |
| 243 | 237 | 238 | 1 | DICT |
| 244 | 230 | 231 | 1 | |
| 245 | 228 | 230 | am | BACKBONE\|DICT\|INTERRES |
| 246 | 247 | 245 | 1 | BACKBONE\|DICT |
| 247 | 247 | 249 | 1 | DICT |
| 248 | 247 | 260 | 1 | BACKBONE\|DICT |
| 249 | 249 | 252 | 1 | DICT |
| 250 | 252 | 255 | 1 | DICT |
| 251 | 255 | 257 | am | DICT |
| 252 | 255 | 256 | 2 | DICT |
| 253 | 247 | 248 | 1 | BACKBONE\|DICT |
| 254 | 249 | 251 | 1 | DICT |
| 255 | 249 | 250 | 1 | DICT |
| 256 | 252 | 254 | 1 | DICT |
| 257 | 252 | 253 | 1 | DICT |
| 258 | 257 | 258 | 1 | DICT |
| 259 | 257 | 259 | 1 | DICT |
| 260 | 245 | 246 | 1 | |
| 261 | 260 | 261 | 2 | |
| 262 | 260 | 262 | 2 | |
| 263 | 243 | 245 | am | BACKBONE\|DICT\|INTERRES |

@<TRIPOS>SUBSTRUCTURE

| | | | | | | | | |
|---|---|---|---|---|---|---|---|---|
| 1 | PHE1 | 1 | RESIDUE | 1 | A | PHE | 1 | ROOT |
| 2 | GLY2 | 25 | RESIDUE | 1 | A | GLY | 2 | |
| 3 | GLY3 | 32 | RESIDUE | 1 | A | GLY | 2 | |
| 4 | PHE4 | 39 | RESIDUE | 1 | A | PHE | 2 | |
| 5 | THR5 | 59 | RESIDUE | 1 | A | THR | 2 | |
| 6 | GLY6 | 73 | RESIDUE | 1 | A | GLY | 2 | |
| 7 | ALA7 | 80 | RESIDUE | 1 | A | ALA | 2 | |
| 8 | ARG8 | 90 | RESIDUE | 1 | A | ARG | 2 | |
| 9 | LYS9 | 114 | RESIDUE | 1 | A | LYS | 2 | |
| 10 | SER10 | 136 | RESIDUE | 1 | A | SER | 2 | |
| 11 | ALA11 | 147 | RESIDUE | 1 | A | ALA | 2 | |
| 12 | ARG12 | 157 | RESIDUE | 1 | A | ARG | 2 | |
| 13 | LYS13 | 181 | RESIDUE | 1 | A | LYS | 2 | |
| 14 | LEU14 | 203 | RESIDUE | 1 | A | LEU | 2 | |
| 15 | ALA15 | 222 | RESIDUE | 1 | A | ALA | 2 | |
| 16 | GLU16 | 232 | RESIDUE | 1 | A | GLU | 2 | |
| 17 | GLN17 | 247 | RESIDUE | 1 | A | GLN | 1 | |

@<TRIPOS>SET
UNK_ATOMS STATIC ATOMS AMSOM **** Atom types
guessed for these atoms
2 261 262
@<TRIPOS>ROTATABLE_BOND
23 24 29 1 1 0 0
@<TRIPOS>ANCHOR_ATOM
25
@<TRIPOS>NORMAL

TABLE 5
@<TRIPOS>MOLECULE
NMR-nociceptin-phe1
17 16 3 6 7
PROTEIN
NO_CHARGES
@<TRIPOS>DICT
PROTEIN PROTEIN
@<TRIPOS>ATOM

| | | | | | | | | | |
|---|---|---|---|---|---|---|---|---|---|
| 1 | CG | 24.6920 | −4.0430 | 7.5260 | C.ar | 1 | <1> | 0.0000 | DICT |
| 2 | CD1 | 25.0490 | −5.2650 | 8.1080 | C.ar | 1 | <1> | 0.0000 | DICT |
| 3 | CD2 | 25.6590 | −3.2710 | 6.8700 | C.ar | 1 | <1> | 0.0000 | DICT |
| 4 | CE1 | 26.3720 | −5.7160 | 8.0340 | C.ar | 1 | <1> | 0.0000 | DICT |
| 5 | CE2 | 26.9820 | −3.7220 | 6.7960 | C.ar | 1 | <1> | 0.0000 | DICT |
| 6 | CZ | 27.3390 | −4.9450 | 7.3780 | C.ar | 1 | <1> | 0.0000 | DICT |
| 7 | CG | 13.4180 | −1.1060 | 9.8800 | C.ar | 2 | PHE4 | 0.0000 | DICT |
| 8 | CD1 | 12.3140 | −0.5000 | 10.4910 | C.ar | 2 | PHE4 | 0.0000 | DICT |
| 9 | CD2 | 14.0960 | −2.1440 | 10.5320 | C.ar | 2 | PHE4 | 0.0000 | DICT |
| 10 | CE1 | 11.8870 | −0.9300 | 11.7530 | C.ar | 2 | PHE4 | 0.0000 | DICT |
| 11 | CE2 | 13.6690 | −2.5750 | 11.7940 | C.ar | 2 | PHE4 | 0.0000 | DICT |
| 12 | CZ | 12.5640 | −1.9680 | 12.4040 | C.ar | 2 | PHE4 | 0.0000 | DICT |
| 13 | CZ | 6.3710 | 0.3180 | 6.1050 | C.2 | 3 | ARG8 | 0.0000 | DICT |
| 14 | NH1 | 6.8590 | 0.9860 | 7.1150 | N.am | 3 | ARG8 | 0.0000 | DICT |
| 15 | HH11 | 7.6500 | 1.5830 | 6.9830 | H | 3 | ARG8 | 0.0000 | DICT\|ESSENTIAL |
| 16 | HH12 | 6.4420 | 0.9000 | 8.0200 | H | 3 | ARG8 | 0.0000 | DICT\|ESSENTIAL |
| 17 | NH2 | 5.3380 | −0.4610 | 6.2770 | O.2 | 3 | ARG8 | 0.0000 | DICT |

@<TRIPOS>BOND

| | | | | |
|---|---|---|---|---|
| 1 | 1 | 2 | ar | DICT |
| 2 | 1 | 3 | ar | DICT |
| 3 | 3 | 5 | ar | DICT |
| 4 | 5 | 6 | ar | DICT |
| 5 | 6 | 4 | ar | DICT |
| 6 | 2 | 4 | ar | DICT |
| 7 | 7 | 8 | ar | DICT |
| 8 | 7 | 9 | ar | DICT |
| 9 | 9 | 11 | ar | DICT |
| 10 | 11 | 12 | ar | DICT |
| 11 | 12 | 10 | ar | DICT |
| 12 | 10 | 8 | ar | DICT |
| 13 | 13 | 17 | 2 | DICT |
| 14 | 13 | 14 | am | DICT |
| 15 | 14 | 15 | 1 | DICT |
| 16 | 14 | 16 | 1 | DICT |

@<TRIPOS>SUBSTRUCTURE

| | | | | | | | | |
|---|---|---|---|---|---|---|---|---|
| 1 | **** | 1 | TEMP | 0 | **** | **** | 0 | ROOT |
| 2 | PHE4 | 7 | RESIDUE | 1 | A | PHE | 0 | ROOT |
| 3 | ARG8 | 13 | RESIDUE | 1 | A | ARG | 0 | ROOT |

@<TRIPOS>SET

| | | | | |
|---|---|---|---|---|
| LAB$A_HETERO | STATIC | ATOMS | LABELGROUP | SYSTEM |

66 0 0 0 0 0 1 2 0 3 0 4 0 5 0 6 0 0 0 0 \
0 0 0 0 0 0 0 0 0 0 7 8 0 9 0 10 0 11 \
0 12 0 0 0 0 0 0 0 0 0 0 0 0 0 0 0 0 \
0 0 13 14 15 16 17 0 0 0 0

| | | | | |
|---|---|---|---|---|
| ATOM$MAGENTA | STATIC | ATOMS | COLORGROUP | SYSTEM |

66 0 0 0 0 0 1 2 0 3 0 4 0 5 0 6 0 0 0 0 \
0 0 0 0 0 0 0 0 0 0 7 8 0 9 0 10 0 11 \
0 12 0 0 0 0 0 0 0 0 0 0 0 0 0 0 0 0 \
0 0 13 14 15 16 17 0 0 0 0

| | | | | |
|---|---|---|---|---|
| UCENT$CENT1 | STATIC | ATOMS | UNITY | SYSTEM\|DELETE_EMPTY |
| 6 1 2 3 4 5 6 | | | | |
| UCENT$CENT2 | STATIC | ATOMS | UNITY | SYSTEM\|DELETE_EMPTY |
| 6 7 8 9 10 11 12 | | | | |
| udst1$DIST3 | STATIC | ATOMS | UNITY | SYSTEM\|DELETE_EMPTY |
| 1 13 | | | | |
| udst1$DIST2 | STATIC | ATOMS | UNITY | SYSTEM\|DELETE_EMPTY |
| 1 13 | | | | |
| LAB$A_TYPE | STATIC | ATOMS | LABELGROUP | SYSTEM |

17 1 2 3 4 5 6 7 8 9 10 11 12 13 14 15 16 17
@<TRIPOS>NORMAL
@<TRIPOS>U_FEAT

| | | | | | | |
|---|---|---|---|---|---|---|
| 1 0 | CENT1 | 1 3 0 | | | | |
| 1 0 | CENT2 | 1 4 0 | | | | |
| 2 6 | DIST1 | 13.860000 | 2.000000 | 0 2 | CENT2 | CENT1 |
| 2 6 | DIST2 | 20.270000 | 4.000000 | 1 6 1 | CENT1 | |
| 2 6 | DIST3 | 8.520000 | 2.000000 | 1 5 1 | CENT2 | |

TABLE 6
@<TRIPOS>MOLECULE
NMR-nociceptin-phe1
15 14 3 6 7
PROTEIN
NO_CHARGES
@<TRIPOS>DICT
PROTEIN PROTEIN
@<TRIPOS>ATOM

| | | | | | | | | | |
|---|---|---|---|---|---|---|---|---|---|
| 1 | CG | 24.6920 | −4.0430 | 7.5260 | C.ar | 1 | <1> | 0.0000 | DICT |
| 2 | CD1 | 25.0490 | −5.2650 | 8.1080 | C.ar | 1 | <1> | 0.0000 | DICT |
| 3 | CD2 | 25.6590 | −3.2710 | 6.8700 | C.ar | 1 | <1> | 0.0000 | DICT |
| 4 | CE1 | 26.3720 | −5.7160 | 8.0340 | C.ar | 1 | <1> | 0.0000 | DICT |
| 5 | CE2 | 26.9820 | −3.7220 | 6.7960 | C.ar | 1 | <1> | 0.0000 | DICT |
| 6 | CZ | 27.3390 | −4.9450 | 7.3780 | C.ar | 1 | <1> | 0.0000 | DICT |
| 7 | CG | 13.4180 | −1.1060 | 9.8800 | C.ar | 2 | PHE4 | 0.0000 | DICT |
| 8 | CD1 | 12.3140 | −0.5000 | 10.4910 | C.ar | 2 | PHE4 | 0.0000 | DICT |
| 9 | CD2 | 14.0960 | −2.1440 | 10.5320 | C.ar | 2 | PHE4 | 0.0000 | DICT |
| 10 | CE1 | 11.8870 | −0.9300 | 11.7530 | C.ar | 2 | PHE4 | 0.0000 | DICT |
| 11 | CE2 | 13.6690 | −2.5750 | 11.7940 | C.ar | 2 | PHE4 | 0.0000 | DICT |
| 12 | CZ | 12.5640 | −1.9680 | 12.4040 | C.ar | 2 | PHE4 | 0.0000 | DICT |
| 13 | NH1 | 6.8590 | 0.9860 | 7.1150 | N.3 | 3 | <3> | 0.0000 | DICT |
| 14 | HH11 | 7.6500 | 1.5830 | 6.9830 | H | 3 | <3> | 0.0000 | DICT\|ESSENTIAL |
| 15 | HH12 | 6.4420 | 0.9000 | 8.0200 | H | 3 | <3> | 0.0000 | DICT\|ESSENTIAL |

@<TRIPOS>BOND

| | | | | |
|---|---|---|---|---|
| 1 | 1 | 2 | ar | DICT |
| 2 | 1 | 3 | ar | DICT |
| 3 | 3 | 5 | ar | DICT |
| 4 | 5 | 6 | ar | DICT |
| 5 | 6 | 4 | ar | DICT |
| 6 | 2 | 4 | ar | DICT |
| 7 | 7 | 8 | ar | DICT |
| 8 | 7 | 9 | ar | DICT |
| 9 | 9 | 11 | ar | DICT |
| 10 | 11 | 12 | ar | DICT |
| 11 | 12 | 10 | ar | DICT |
| 12 | 10 | 8 | ar | DICT |
| 13 | 13 | 14 | 1 | DICT |
| 14 | 13 | 15 | 1 | DICT |

@<TRIPOS>SUBSTRUCTURE

| | | | | | | | | |
|---|---|---|---|---|---|---|---|---|
| 1 | **** | 1 | TEMP | 0 | **** | **** | 0 | ROOT |
| 2 | PHE4 | 7 | RESIDUE | 1 | A | PHE | 0 | ROOT |
| 3 | **** | 13 | TEMP | 0 | **** | **** | 0 | ROOT |

@<TRIPOS>SET

| | | | | |
|---|---|---|---|---|
| LAB$A_HETERO | STATIC | ATOMS | LABELGROUP | SYSTEM |

17 1 2 3 4 5 6 7 8 9 10 11 12 0 13 14 15 0

| | | | | |
|---|---|---|---|---|
| ATOM$MAGENTA | STATIC | ATOMS | COLORGROUP | SYSTEM |

66 0 0 0 0 0 1 2 0 3 0 4 0 5 0 6 0 0 0 0 \
0 0 0 0 0 0 0 0 0 0 7 8 0 9 0 10 0 11 \
0 12 0 0 0 0 0 0 0 0 0 0 0 0 0 0 0 0 \
0 0 0 13 14 15 0 0 0 0 0

| | | | | |
|---|---|---|---|---|
| UCENT$CENT1 | STATIC | ATOMS | UNITY | SYSTEM\|DELETE_EMPTY |
| 6 1 2 3 4 5 6 | | | | |
| UCENT$CENT2 | STATIC | ATOMS | UNITY | SYSTEM\|DELETE_EMPTY |
| 6 7 8 9 10 11 12 | | | | |
| udst1$DIST2 | STATIC | ATOMS | UNITY | SYSTEM\|DELETE_EMPTY |
| 1 13 | | | | |
| udst1$DIST3 | STATIC | ATOMS | UNITY | SYSTEM\|DELETE_EMPTY |
| 1 13 | | | | |
| LAB$A_TYPE | STATIC | ATOMS | LABELGROUP | SYSTEM |

17 1 2 3 4 5 6 7 8 9 10 11 12 0 13 14 15 0
@<TRIPOS>NORMAL
@<TRIPOS>U_FEAT

| | | | | | | |
|---|---|---|---|---|---|---|
| 1 0 | CENT1 | 1 3 0 | | | | |
| 1 0 | CENT2 | 1 4 0 | | | | |
| 2 6 | DIST1 | 13.860000 | 2.000000 | 0 2 | CENT2 | CENT1 |
| 2 6 | DIST2 | 19.930000 | 4.000000 | 1 5 1 | CENT1 | |
| 2 6 | DIST3 | 7.760000 | 2.000000 | 1 6 1 | CENT2 | |

Opioid Receptor Binding and Calcium Release. Table 3 gives results for ORL1 receptor binding (Kᵢ values) and calcium release (EC₅₀ values) for nociceptin and 30 single and double amino acid-substituted variants.

TABLE 3
Binding of nociceptin (NC) [F¹GGFT⁵GARKS¹⁰ARKLA¹⁵NQ¹⁷] peptides
to the nociceptin receptor, and functional activity (calcium release) of
substituted nociceptin peptides.

| Peptide | Kᵢ[nM] | Kᵢ(x)/Kᵢ(WT) | EC50 [nM] | EC50(x)/EC50(WT) |
|---|---|---|---|---|
| nociceptin | 0.7* | 1.0 | 2.4* | 1.0 |
| F1A | 91 | 127 | 1443 | 601 |
| G2A | 191 | 265 | 1394 | 581 |
| G3A | 1.6 | 2.2 | 13 | 5.5 |
| F4A | 188 | 262 | 3881 | 1617 |
| T5A | 6.4 | 8.9 | 159 | 66 |
| G6A | 1.5 | 2.1 | 39 | 16 |
| A7G | 7.7 | 11 | 18 | 7.4 |
| R8A | 50 | 69 | 69 | 29 |
| K9A | 1.5 | 2.1 | 2.8 | 1.2 |
| S10A | 0.8 | 1.2 | 42 | 18 |
| A11G | 3.2 | 4.4 | 11 | 4.8 |
| R12A | 2.8 | 3.9 | 1.3 | 0.5 |
| K13A | 1.5 | 2.1 | 1.1 | 0.5 |
| L14A | 0.2 | 0.3 | 7.0 | 2.9 |
| A15G | 0.2 | 0.3 | 1.9 | 0.8 |
| N16A | 0.3 | 0.4 | 1.7 | 0.7 |
| Q17A | 0.8 | 1.1 | 12 | 4.8 |
| T5L | 2.2 | 3.1 | 14 | 5.7 |
| T5S | 1.4 | 2.0 | 27 | 11 |
| A7L | 0.8 | 1.1 | 13 | 5.2 |
| A7nleu | 0.5 | 0.7 | 29 | 12 |
| A7S | 1.5 | 2.1 | 5.6 | 2.3 |
| R8cit | 31 | 43 | 19 | 7.7 |
| R8K | 32 | 45 | 64 | 27 |
| A11L | 0.9 | 1.2 | 8.3 | 3.5 |
| A11nleu | 1.9 | 2.6 | 4.2 | 1.8 |
| A11S | 1.2 | 1.7 | 1.2 | 0.5 |
| R12cit | 0.8 | 1.1 | 1.3 | 0.5 |
| R12K | 0.8 | 1.1 | 2.4 | 1.0 |
| R8K/ | 3.9 | 5.4 | 53.5 | 22 |
| R12K | | | | |

*Results depicted are from experiments repeated 3 times, in duplicate. Standard deviations range generally from 5% to 10% of the value reported.

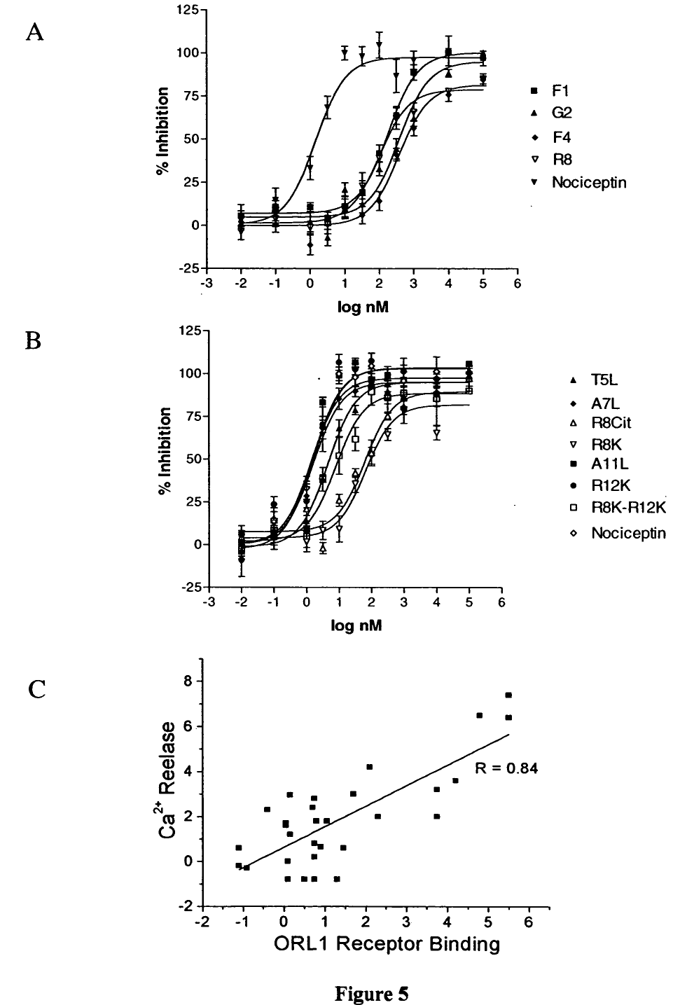

The first 18 rows provide results from alanine (or glycine when an alanine was already present) scanning, while the last 13 rows show results from various other substitutions to better discern effects from changes in charge, polarity and side-chain composition. Representative binding curves of alanine and glycine substitutions at critical residues are shown in FIG. 5B. The ability of the different substituted peptides to bind to ORL1 and to function as agonists as assessed by calcium release correlate well, with a correlation coefficient of 0.78 (FIG. 5C).

Alanine substitutions of residues at positions 1 and 4 produced some of the most dramatic decreases in receptor binding and calcium release (Table 3 and FIG. 5A). This was anticipated because previous studies reported that these residues are in the N-terminal “address” portion of the molecule known to be critical for ORL1 receptor binding (Reinscheid et al., J. Biol. Chem., 271, 14163-14168 (1996); Butour et al., Eur. J. Pharmacol., 321, 97-103 (1997); Guerrini et al., J. Med. Chem., 40, 1789-1793 (1997)). It was surprising, however, that other alanine-substituted variants also produced relatively large effects. In particular, substitution of G2 for alanine (G2A) produced the largest single drop in binding affinity, twice that of substitution of F1. One explanation for this is induced conformational changes in the peptide when alanine is present at position 2, as opposed to the greater freedom of rotation around the peptide bond provided by the glycine residue. Another sensitive position is at R8, where substitution with alanine led to a significant decrease in binding affinity (FIG. 5A) and signaling (as evidenced by calcium release). Substitution of neighboring A7 with glycine also caused a substantial reduction in binding affinity. Modest effects were also noted with substitutions of A11 and R12 on receptor binding and of S10 and A11 on signaling. In addition, substitutions at C-terminal residues L14, A15, and N16, which have been reported to be dispensible for signalling, produced a modest 2- to 3-fold increase in receptor binding affinity, while signaling remained unaffected with substitutions at A15 and N16. Modifications at the C-terminal end of the peptide may provide a way to design a more potent antagonist for nociceptin.

Substitution of T5 with leucine (FIG. 5B) or serine was better tolerated than substitution with alanine, suggesting that side-chain bulkiness and/or structure-inducing properties were important to the bioactive conformation of the peptide. Similarly, replacement of A7 with leucine, norleucine or serine produced little if any change on receptor binding (FIG. 5B) and only a modest change in functional activity, expect for norleucine which demonstrated a 12-fold decrease in signaling (Table 3). Apparently, the presence of an unbranched butyl group at position 7 has a negative effect on function, but not on receptor binding. At position 11, addition of a β-hydroxy group (serine) at position 11 was well tolerated, whereas replacement of that alanine with bulkier leucine or norleucine residues had a modest negative effect on receptor binding and signaling (FIG. 5B and Table 3).

Substitution of the basic arginine with lysine at position 8 was not well tolerated in terms of both receptor binding and calcium release, while substitution of R8 with cirtrulline also had a negative effect on receptor binding, but showed a much more modest effect on signaling (FIG. 5B and Table 3). A different trend was noted for the arginine at position 12, where substitution with alanine had a modest effect on receptor binding, but essentially no effect on signaling. Substitution with lysine or citrulline had essentially no effect on binding or function. Simultaneous substitution of both arginines at position 8 and 12 with lysine produced a peptide that behaved similarly to the R8A substitution alone with respect to signal, i.e., calcium release was reduced to a similar extent (FIG. 5B and Table 3). This is in contrast to the behavior of these peptides in the receptor binding assay, where lysine substitution at both R8 and R12 produced a molecule that bound with an affinity closer to that of the wild-type peptide (FIG. 5B). Taken together, these findings suggest that the presence of positive charges at positions 8 and 12 is not the sole determining factor for potency. The hydrogen-bonding potential of arginine compared to that of lysine may, at least partly, help explain this observation.

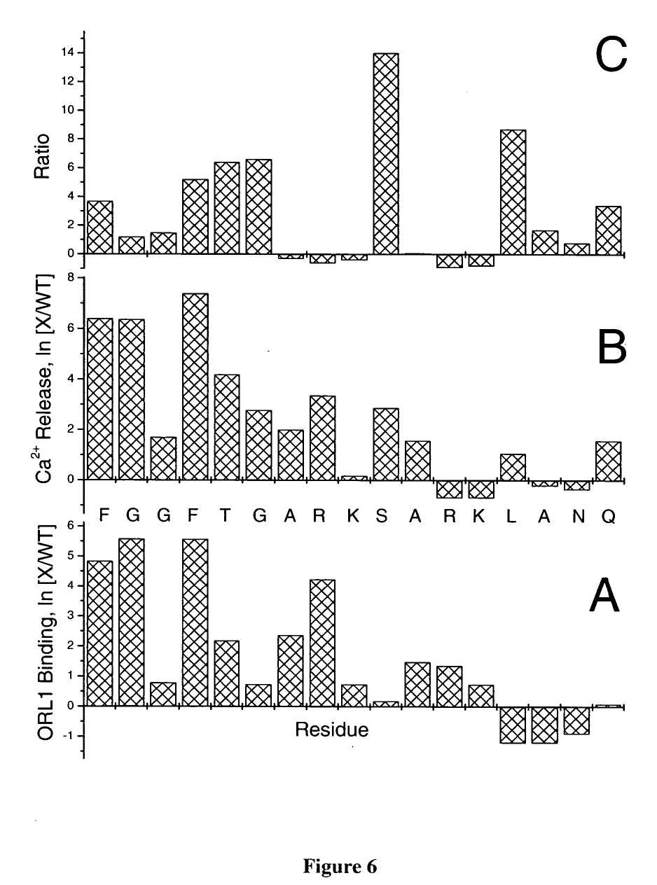

Nociceptin Structure-Function Relationships. Based on NMR data and structural modeling, nociceptin in SDS micelles forms a helix from residue F4 to the C-terminus. While one can not conclude definitively from these biophysical data that a helix is the bioactive conformation for nociceptin, further analysis of the alanine/glycine scanning data for ORL1 receptor binding and signaling strongly supports this view. By plotting the ratios of the binding and functional activities of the nociceptin mutant peptides to those of the native peptide, the helical periodicity becomes apparent (FIGS. 6A and 6B). Although both receptor binding and calcium release data show this trend, it is more evident with the receptor binding data. Starting at F4 and moving towards the C-terminus, substitutions made at every (i+3) or (i+4) residue have a deliterious effect on activity. This effect diminishes in magnitude as one moves closer to the C-terminus, consistent with residues at the N-terminus being most important to binding of nociceptin to ORL1 receptor.

The nociceptin peptide structure-function relationships described here allow for refinement of a pharmacophore model based on nociceptin residues important for binding and signaling through ORL1. Such a model is expected to aid in identifying novel non-peptide modulators of the ORL1 receptor as potential drug candidates. The pharmacophore site for nociceptin has the phenyl ring of F1 on top of that for F4. The first turn of the helix then places F4 over A7 and R8, followed by the second turn of the helix with another Ala-Arg pair, A11 and R12. This fold causes the two pairs of alanine-arginine residues, both of which appear to be crucial to activity, to lie on top of one another. This could mean that nociceptin, when folded in this fashion, interacts with ORL1 via both hydrophobic residues and positively charged residues. As mentioned above, it also appears that the hydrogen bonding potential of the arginine guanidinium group, and not only its positive charge, plays a role in the activity of nociceptin. While both the R8K and R8cit variants show significant losses in their receptor binding activities, calcium release is relatively less affected by substitution with the polar, yet neutral, citrulline moiety.

There is clearly a relationship between receptor binding and signal transduction). However, the correlation shown in FIG. 5C is not absolute. In fact, substitution of one residue may have a greater effect on signal transduction than on receptor binding and vice versa. The ratio of signaling to binding is plotted in FIG. 6C. The larger and more positive this ratio is, the larger the effect of the substitution on signal transduction relative to ORL1 binding. As expected, reasonably good effects are noted at the N-terminal “address” portion of nociceptin. However, even within this region, it appears that the largest effects are found with T5 and G6, suggesting that these two residues may be somewhat more crucial to signal transduction than to receptor binding relative, for example, to F1 and F4. What is most surprising in this plot is that C-terminal residues S10, L14 and Q17 also display greater capacity to affect signal transduction. It has been reported that these C-terminal residues are dispensible for nociceptin activity (Guerrini et al., J. Med. Chem., 40, 1789-1793 (1997); Butour et al., Eur. J. Pharmacol., 321, 97-103 (1997)); however, using through the lens of this signaling-to-binding ratio, it appears that the nociceptin C-terminus does play a role in ORL1 receptor signaling. Note also that the activity profile for these residues also shows (i+3, i+4) helix periodicity. From this analysis, one can also conclude that the side-chain of S10 as well plays a relatively important role in signaling, but not in ORL1 receptor binding. This perspective on these data may aid in designing nociceptin mimetics as agonists or as antagonists. For example, an antagonist would be excellent at receptor binding, but poor at signal transduction.

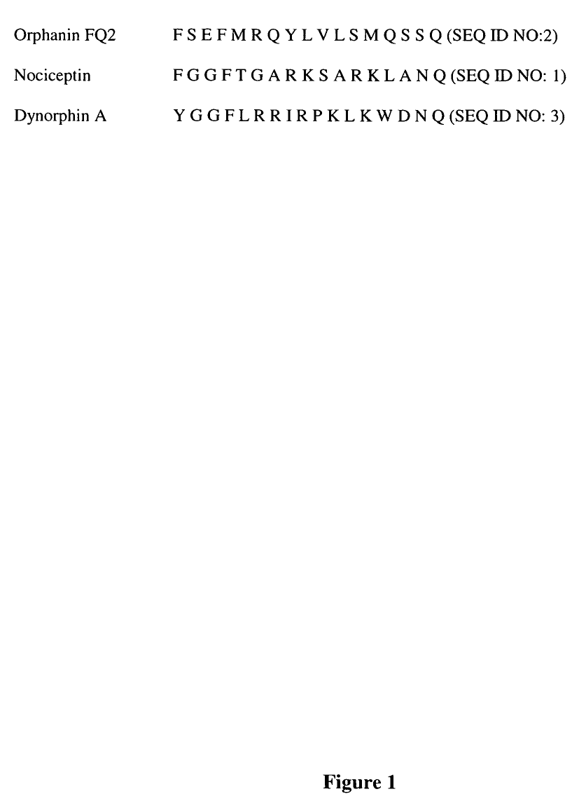

Comparison to Related Peptides. As shown in FIG. 1, dynorphin A and orphanin FQ2 are homologous to nociceptin. To test the intrinsic tendency of dynorphin A to assume helical conformations, Spadaccini et al., J. Peptide Sci., 5, 306-312 (1999), studied the conformational properties of dynorphin A over a range of solution conditions: methanol, acetonitrile, DMSO and mixtures of organic solvents in water. In most of these solutions, the peptide showed conformational flexibility comparable to that observed in water alone, i.e., no stable structure. In DMSO/water (80:20), however, some structural stabilization was noted from R7 to Q17. Nevertheless, the peptide demonstrated little regular structure. Amodeo et al., FEBS Letters, 473, 157-160 (2000), investigated the NMR solution structure of orphanin FQ2 and concluded similarly that it too possess helix conformation through the body of the peptide. Obviously, receptor binding and function are somewhat different for each of these peptides. One may conclude that not only the amino acid sequence, but also the spatial relations of residues, account for different activities relative to nociceptin. In a helix conformation, amino acid residues would be presented to the receptor differently in each of these homologous peptides. This, in turn, would define different pharmacophore sites in each peptide.

Small Molecule Database Search using the Pharmacophore Model. To demonstrate that the pharmacophore model, which was based on the NMR-derived structure of the peptide and SAR information, could lead to hits with demonstrated nociceptin receptor binding affinities, a three-point pharmacophore query was made. This query contained side chain functionalities from the F1 aromatic, the F4 aromatic and the R8 terminal nitrogen of the guanidinium group. These three amino acid residues were clearly important for binding and function. The pharmacophore was used to search a database of 250,000 small molecule structures and 940 unique compounds were obtained that fit the model. Representative compounds are shown in FIG. 7.

These compounds were tested in duplicate in the receptor binding assay at a concentration of 10 μM. Of these compounds, 37 inhibited the binding of labeled ¹²⁵I-nociceptin by 50% or more, which represents a hit rate of 3.9%. Random high throughput screening of the same compound library at a screening concentration of 16 μM yielded 1,139 hits (defined as inhibition of 50% or more) out of 188,787 compounds, a hit rate of 0.6%. Fifteen compounds that were determined to inhibit the binding of labeled ¹²⁵I-nociceptin by 50% or more at a concentration of 10 μM are shown in FIG. 7.

Thus, the use of our pharmacophore model to select compounds increased the hit rate by nearly 7-fold from a library that was 200-fold smaller than the size of a random library. These results indicate that a pharmacophore-based selection process can significantly reduce the time and effort expended to find lead compounds, which can be of even greater value for peptide receptors, where initial small molecule leads may be difficult to find.

Conclusions. This study has demonstrated that structure-activity relationships in noniceptin, deduced using NMR-derived structures and functional assays, can lead to identification of a more complete pharmacophore site. The approach has been validated by using elements from this pharmacophore site in a 3D database search for small molecule hits that can function like nociceptin. This will aid in the design of a clinically useful pharmaceutical drug for nociceptin.

The complete disclosures of all patents, patent applications, publications, and nucleic acid and protein database entries, including for example GenBank accession numbers and EMBL accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

1. A method of determining a nociceptin pharmocophore, the method comprising:
collecting nuclear magnetic resonance (NMR) data of nociceptin or a peptide analog thereof in an aqueous composition;
determining distances and angles between atoms of nociceptin from the NMR data;
computer modeling the three-dimensional structure of nociceptin in the aqueous composition based on the NMR data; and
conducting a structure-activity analysis to identify pharmacophore elements of the solution structure of nociceptin.

2. The method of claim 1 wherein collecting NMR data comprises carrying out a multidimensional NMR experiment on the aqueous composition of nociceptin.

3. The method of claim 2 wherein the multidimensional NMR experiment is a 2D homonuclear NMR experiment.

4. The method of claim 1 wherein determining distances and angles from NMR data comprises carrying out Nuclear Overhauser Effect (NOE) experiments.

5. The method of claim 1 wherein the aqueous composition comprises a membrane-like environment.

6. The method of claim 5 wherein the membrane-like environment comprises a hydrophobic compound.

7. The method of claim 6 wherein the hydrophobic compound is used in an amount sufficient to provide an aqueous composition having a dielectric constant of less than 40.

8. The method of claim 1 wherein conducting a structure-activity analysis comprises conducting an amino acid scan.

9. The method of claim 1 wherein the nociceptin pharmacophore comprises at least three elements comprising two hydrophobic features and one polar feature or a feature capable of electrostatic interaction.

10. The method of claim 9 wherein the nociceptin pharmacophore comprises at least three elements comprising two hydrophobic features and one positively charged feature or a feature capable of hydrogen bonding.

11. The method of claim 10 wherein the hydrophobic features are aromatic rings.

12. The method of claim 10 wherein the pharmacophore comprises the side chain functionalities from the Phe-1 aromatic group, the Phe-4 aromatic group, and the Arg-8 terminal nitrogen atom of the guanidinium group.

13. The method of claim 1 wherein a three-dimensional structure of nociceptin is represented by the Cartesian coordinates listed in Table 4.

14. The method of claim 1 wherein a three-dimensional structure of a nociceptin pharmacophore is represented by the Cartesian coordinates listed in Table 5.

15. The method of claim 1 wherein a three-dimensional structure of a nociceptin pharmacophore is represented by the Cartesian coordinates listed in Table 6.

16. A method of identifying a nociceptin mimic, the method comprising:
determining a nociceptin pharmocophore comprising:
collecting nuclear magnetic resonance (NMR) data of nociceptin in an aqueous composition;
determining distances and angles between atoms and features of nociceptin from the NMR data;
computer modeling the three-dimensional structure of nociceptin in the aqueous composition based on the NMR data; and
conducting a structure-activity analysis to identify pharmacophore elements of the solution structure of nociceptin;
supplying a three-dimensional structure of a test compound;
comparing the structural features of a test compound to the pharmacophore to determine if it is a potential nociceptin mimic; and
evaluating the binding capacity of the potential mimic to a nociceptin receptor, wherein a nociceptin mimic inhibits the binding of labeled ¹²⁵I-nociceptin to human nociceptin receptor (ORL-1) on HEK-293 cell membranes by 50% or more at a concentration of 10 μM.

17. The method of claim 16 wherein evaluating the binding capacity comprises using computer modeling techniques to evaluate the potential ability of the potential mimic to bind to a nociceptin receptor.

18. The method of claim 17 wherein evaluating the binding capacity further comprises subjecting the potential mimic to a binding assay.

19. The method of claim 16 wherein collecting NMR data comprises carrying out a 2D homonuclear NMR experiment on the aqueous composition of nociceptin, wherein the aqueous composition comprises a membrane-like environment.

20. The method of claim 16 wherein determining distances and angles from NMR data comprises carrying out Nuclear Overhauser Effect (NOE) experiments.

21. The method of claim 16 wherein conducting a structure-activity analysis comprises conducting an amino acid scan.

22. The method of claim 16 wherein the nociceptin pharmacophore comprises at least three elements comprising two hydrophobic features and one polar feature or a feature capable of electrostatic interaction.

23. The method of claim 22 wherein the nociceptin pharmacophore comprises at least three elements comprising two hydrophobic features and one positively charged feature of a feature capable of hydrogen bonding.

24. The method of claim 23 wherein the pharmacophore comprises the side chain functionalities from the Phe-1 aromatic group, the Phe-4 aromatic group, and the Arg-8 terminal nitrogen atom of the guanidinium group.

25. A nociceptin pharmacophore comprising at least three elements comprising two hydrophobic features and one polar feature or a feature capable of electrostatic interaction.

26. A nociceptin pharmacophore comprising at least three elements comprising two hydrophobic features and one positively charged feature or a feature capable of hydrogen bonding.

27. The nociceptin pharmacophore of claim 26 wherein the hydrophobic features are aromatic rings.

28. The nociceptin pharmacophore of claim 26 wherein the pharmacophore comprises the side chain functionalities from the Phe-1 aromatic group, the Phe-4 aromatic group, and the Arg-8 terminal nitrogen atom of the guanidinium group.

29. A nociceptin pharmacophore comprising a three-dimensional structure represented by the Cartesian coordinates listed in Table 5.

30. A nociceptin pharmacophore comprising a three-dimensional structure represented by the Cartesian coordinates listed in Table 6.

31. A solution conformation of nociceptin in a membrane-like environment, wherein nociceptin forms a helix, amino acids 5 through 17 of nociceptin are folded aperiodically on top of the helix such that two pairs of alanine-arginine residues lie on top of one another and two pairs of alanine-arginine residues lie on top of one another.

32. The solution conformation of claim 31 wherein the phenyl ring of F1 is positioned on top of that for F4, which is positioned over A7 and R8.

33. The solution conformation of claim 32 wherein the surface active domain comprises amino acids F1, F4, R8, A7, R12, and A11 shown in FIG. 4.

34. A solution conformation of nociceptin comprising a three-dimensional structure represented by the Cartesian coordinates listed in Table 4.

35. A machine-readable data storage medium comprising a data storage material encoded with structure coordinates listed in Table 4.

36. A machine-readable data storage medium comprising a data storage material encoded with structure coordinates listed in Table 5.

37. A machine-readable data storage medium comprising a data storage material encoded with structure coordinates listed in Table 6.

38. A method of identifying a compound that binds to a nociceptin receptor, the method comprising:
determining a nociceptin pharmocophore comprising:
collecting nuclear magnetic resonance (NMR) data of nociceptin in an aqueous composition;
determining distances and angles between atoms and features of nociceptin from the NMR data;
computer modeling the three-dimensional structure of nociceptin in the aqueous composition based on the NMR data; and
conducting a structure-activity analysis to identify pharmacophore elements of the solution structure of nociceptin;
supplying a three-dimensional structure of a test compound;
comparing the structural features of a test compound to the pharmacophore to determine if the test compound will potentially bind to a nociceptin receptor; and
evaluating the binding capacity of the identified test compound to a nociceptin receptor.
